SEQUENCE LISTING (23 sequences)

SEQ ID NO: 1 - protein, 321 aa, Escherichia coli
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEK

SEQ ID NO: 2 - DNA, 2491 bp, Escherichia coli
caaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatccccccacagatacggtaaactagcctcgtttttgcatcaggaaagcagctatgaaccactccttaaaaccctggaacacatttggcattgatcataatgctcagcacattgtatgtgccgaagacgaacaacaattactcaatgcctggcagtatgcaaccgcagaaggacaacccgttcttattctgggtgaaggaagtaatgtactttttctggaggactatcgcggcacggtgatcatcaaccggatcaaaggtatcgaaattcatgatgaacctgatgcgtggtatttacatgtaggagccggagaaaactggcatcgtctggtaaaatacactttgcaggaaggtatgcctggtctggaaaatctggcattaattcctggttgtgtcggctcatcacctatccagaatattggtgcttatggcgtagaattacagcgagtttgcgcttatgttgattctgttgaactggcgacaggcaagcaagtgcgcttaactgccaaagagtgccgttttggctatcgcgacagtatttttaaacatgaataccaggaccgcttcgctattgtagccgtaggtctgcgtctgccaaaagagtggcaacctgtactaacgtatggtgacttaactcgtctggatcctacaacagtaacgccacagcaagtatttaatgcggtgtgtcatatgcgcaccaccaaactccctgatccaaaagtgaatggcaatgccggtagtttcttcaaaaaccctgttgtatctgccgaaacggctaaagcattactgtcacaatttccaacagcaccaaattacccccaggcggatggttcagtaaaactggcagcaggttggcttatcgatcagtgccagctaaaagggatgcaaataggtggggctgcggtgcaccgtcaacaggcgttagttctcattaatgaagacaatgcaaaaagcgaagatgttgtacagctggcgcatcatgtaagacagaaagttggtgaaaaatttaatgtctggcttgagcctgaagtccgctttattggtgcatcaggtgaagtgagcgcagtggagacaatttcatgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgccagtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaataagaaaggggagtattcgctcccctgcaaattatttgcgtagtctgacctcttctaccgcatgattagcacttttcgtcaggattaaactggcgcgctcacgagtaggtagaatattttgctttaagttcagccagttgatctctttccacaatgtcatggcagtcttaatcgcttcttctttagttaatttcgcgtagtta

SEQ ID NO: 3 - protein, 13 aa, Artificial Sequence
  Synthetic peptide
LXXIXXXXKXXXX

SEQ ID NO: 4 - protein, 14 aa, Artificial Sequence
  Synthetic peptide
GLNDIFEAQKEWHE

SEQ ID NO: 5 - protein, 15 aa, Artificial Sequence
  Synthetic peptide
DTLCIVEAMKMMNQI

SEQ ID NO: 6 - protein, 15 aa, Artificial Sequence
  Synthetic peptide
GLNDIFEAQKIEWHE

SEQ ID NO: 7 - protein, 76 aa, Escherichia coli
HIVRSPMVGTFYRTPSPDAKAFIEVGQKVNVGDTLCIVEAMKMMNQIEADKSGTVKAILVESGQPVEFDEPLVVIE

SEQ ID NO: 8 - protein, 76 aa, Pseudomonas aeruginosa
NVVRSPMVGTFYRAASPTSANFVEVGQSVKKGDILCIVEAMKMMNHIEAEVSGTIESILVENGQPVEFDQPLFTIV

SEQ ID NO: 9 - protein, 68 aa, Rattus sp.
SVLRSPKPGVVVAVSVKPGDMVAEGQEICVIEAMKMQNSMTAGKMGKVKLVHCKAGDTVGEGDLLVEL

SEQ ID NO: 10 - protein, 68 aa, Homo sapiens
SVLRSPMPGVVVAVSVKPGDAVAEGQEICVIEAMKMQNSMTAGKTGTVKSVHCQAGDTVGEGDLLVEL

SEQ ID NO: 11 - protein, 69 aa, Saccharomyces cerevisiae
LHIGAPMAGVIVEVKVHKGSLIKKGQPVAVLSAMKMEMIISSPSDGQVKEVFVSDGENVDSSDLLVLLE

SEQ ID NO: 12 - protein, 69 aa, Rattus sp.
GQIGAPMPGKVIDIKVVAGAKVAKGQPLCVLSAMKMETVVTSPMEGTVRKVHVTKDMTLEGDDLILEIE

SEQ ID NO: 13 - protein, 69 aa, Homo sapiens; identical to SEQ ID NO: 12

SEQ ID NO: 14 - protein, 68 aa, Gallus gallus
SILRSPSAGKLIQYVVEDGGHVFAGQCFAEIEVMKMVMTLTAGESGCIHYVKRPGAVLDPGCVIAKLQ

SEQ ID NO: 15 - protein, 68 aa, Homo sapiens
SVMRSPSAGKLIQYIVEDGGHVLAGQCYAEIEVMKMVMTLTAVESGCIGYVKRPGAALDPGCVLAKMQ

SEQ ID NO: 16 - protein, 68 aa, Saccharomyces cerevisiae
TQLKTPSPGKLVKFLVENGEHIIKGQPYAEIEVMKMQMPLVSQENGIVQLLKQPGSTIVAGDIMAIMT

SEQ ID NO: 17 - protein, 69 aa, Propionibacterium shermanii
GEIPAPLAGTVSKILVKEGDTVKAGQTVLVLEAMKMETEINAPTDGKVEKVLVKERDAVQGGQGLIKIG

SEQ ID NO: 18 - protein, 69 aa, Methanococcus jannaschii
GAVTSPFRGMVTKIKVKEGDKVKKGDVIVVLEAMKMEHPIESPVEGTVERILIDEGDAVNVGDVIMIIK

SEQ ID NO: 19 - protein, 17 aa, Artificial Sequence
  Synthetic peptide
MSGLNDIFEAQKIEWHE

SEQ ID NO: 20 - protein, 21 aa, Artificial Sequence
  Synthetic peptide
LERAPGGLNDIFEAQKIEWHE

SEQ ID NO: 21 - protein, 8 aa, Artificial Sequence
  Synthetic peptide
WSHPQFEK

SEQ ID NO: 22 - protein, 15 aa, Artificial Sequence
  Synthetic peptide
GLNDIFEAQKIEWHE

SEQ ID NO: 23 - protein, 21 aa, Artificial Sequence
  Synthetic peptide
KKKGPGGLNDIFEAQKIEWHE

(19) United States
(12) Patent Application Publication
(10) Pub. No.: US 2012/0083599 A1
(43) Pub. Date: Apr. 5, 2012

(54) Biomolecular Labelling Using Multifunctional Biotin Analogues

(75) Inventors: Neil R. Thomas, Nottingham (GB); Yang Yong-Qing, Nottingham (GB); William C. Drewe, Nottingham (GB)

(73) Assignee: UNIVERSITY OF NOTTINGHAM, Nottingham (GB)

(21) Appl. No.: 13/257,562

(22) PCT Filed: Mar. 22, 2010
(86) PCT No.: PCT/GB10/00528

§ 371 Date: Dec. 2, 2011

(30) Foreign Application Priority Data
Mar. 20, 2009 (GB) 0904842.2
Apr. 30, 2009 (GB) 0907430.3

(51) Int. Cl. C07H 19/20; C07D 495/04

(52) U.S. Cl. 536/26.6; 548/303.7; 536/26.7

(57) ABSTRACT

Novel biotin analogues, such as 2-Azidobiotin, comprising the ureido ring of natural biotin with the thiophene ring, optionally modified, and a modified sidechain having a functional end group, preferably selected from the group consisting of a carboxylic acid, amine, alcohol, thiol, aldehyde and a halide, and at least one bio-orthogonally reactive chemical group located elsewhere in the sidechain. The analogues are used for labelling target structures and biomolecules, such as peptides and proteins in vitro or in vivo.

### RELATED APPLICATIONS

This application is a 35 USC §371 of PCT Application Serial No. PCT/GB2010/000528, filed Mar. 22, 2010, currently pending, entitled “Biomolecular Labelling Using Multifunctional Biotin Analogues,” which claims priority to Great Britain Patent Application No. 0904842.2, filed Mar. 20, 2009, entitled “Specific Protein Labelling Using Multifunctional Biotin Analogues,” and further claims priority to Great Britain Patent Application No. 0907430.3, filed Apr. 30, 2009, entitled “Biomolecular Labelling Using Multifunctional Biotin Analogues,” each of which is incorporated herein in its entirety by reference.

Incorporated by reference herein in its entirety is the Sequence Listing entitled “SEQ LSTING_ST 25_{—}14 Jun. 2010.txt”, created Jun. 14, 2010, size of 18 kilobytes.

### DESCRIPTION

The present invention relates to biotin analogues and methods of use thereof for labelling target structures and biomolecules, such as peptides and proteins in vitro or in vivo.

The tracking of protein expression, localization and/or conformational changes as components of cellular signalling pathways, requires the creation of general tools for in vivo site-specific labelling of proteins with fluorophores or other useful probes. Traditional chemical methods rely on the nucleophilicity of cysteine or lysine side chains but are too promiscuous for in vivo use. Genetic methods such as fusion to green fluorescent protein (GFP) carry bulky payloads (GFP is 238 amino acids) and are limited in the colour range and nature of the spectroscopic readout.

A number of methods have been introduced over the last few years for the site-specific addition of small molecular probes on to proteins that bear a small specific peptide sequence, including the TetraCys/FlAsH system(B. A. Griffin, S. R. dams, R. Y. Tsien, Science, 1998, 281, 269-272), the labelling of HexaHis (S. Lata, M. Gavutis, R. Tampe, J. Piehler, JACS, 2006, 128, 2365-2372) and polyAsp tags (H. Nonaka, S. Tsukiji, A. Ojida, I. Hamachi, JACS, 2007, 129, 15777-157779). Several enzyme mediated labelling systems have also been reported including ones that use sortase A/SorTag (M. W. Popp, J. M. Antos, G. M. Grotenbreg, E. Spooner, H. L. Ploegh, Nature Chem. Biol. 2007, 3, 707-708; T. Tanaka, T. Yamamoo, S. Tsukiji, T. Nagmune, CemBioChem, 2008, 9, 802-807), transglutaminase/Q-tag (C. W. Lin, A. Y. Ting, JACS, 2006, 128, 4542-4543), biotin ligase (I. Chen, M. Howarth, W. Lin, A. Y. Ting, Nature Methods, 2005, 2, 99-104; M. Howarth, K. Takao, Y. Hyashi, A. Y. Ting, PNAS, 2005, 102, 7583-7588; M. Howarth et al. Nature Methods, 2006, 3, 267-273) and lipoic acid ligase (M. Fernandez-Suarez et al. Nature Biotech. 2007, 25, 1483-1487; H. Baruh, S. Puthebveetil, Y. A. Choi, S. Shah, A. Y. Ting, Angew. Chem. Int. Ed. Engl. 2008, 47, 7018-7021).

Many natural enzymes have evolved marked substrate specificity to fulfill their biological functions. One example is E. coli enzyme biotin ligase (BirA) which participates in the transfer of CO₂ from bicarbonate to organic acids to form various cellular metabolites. (Chapman-Smith et al. J. Nutr. 129:477 S-484S, 1999.) It has only one natural substrate in bacteria: the biotin carboxyl carrier protein (BCCP), which it biotinylates at lysine 122 to prepare it for carboxylation by bicarbonate. Schatz et al. used peptide panning to identify a minimal, 15-amino acid peptide sequence that could be recognized and enzymatically biotinylated by BirA (Schatz et al. Biotechnology 11:1138-1143, 1993; Beckett et al. Protein Sci. 8:921-929, 1999.) The 15 amino acid sequence TTNWVAQAFKMTFDP (SEQ. ID No. 19) is the most efficient 15 amino acid acceptor peptide sequence identified for yeast biotin ligase from a phage display library (I. Chen, Y.-A. Choi and A. Y. Ting, J. Am. Chem. Soc. 2007, 129, 6619). Purified BirA and cloning vectors for introducing this modification sequence, called “Avi-Tag™” onto proteins of interest for site-specific biotinylation in vitro or in living bacteria are commercially available. (Avidity, Boulder, Colo. USA) as is a 72-amino acid sequence from the K. pneumoniae BCCP, supplied under the trade name BioEase™ by Invitrogen. The BioEase™ Expression System provides a method for expressing, purifying and detecting biotinylated recombinant proteins. The BioEase™ vectors include a 72 amino acid sequence from K. pneumoniae oxaloacetate decarboxylase that directs in vivo biotinylation of a specific lysine residue. Proteins produced in the vectors are expressed as fusion proteins with this sequence.

Biotin (Vitamin H or B7) and analogues thereof have also been previously described in relation to the labelling of peptides and proteins in vitro or in vivo. BirA is known to be able to highly selectively attach ketone biotin (Chen et al Nature Meth. 2005, 2, 99-104; McNeill et al. Organic Lett. 2006 8, 4593-4595) to the alpha-amino group of a lysine included in a specific 15 amino acid sequence. The problem with the use of ketone biotin is that its ketone group is relatively reactive in the absence of the enzyme BirA, causing it to react with lysine side chains on the biotin ligase or on other proteins present in the reaction system. Furthermore, the compound was found to give a product that inhibited BirA ligation yields to ˜50%.

Other biotin analogues have been prepared. Ting et al have prepared, inter alia, desthiobiotin azide, cis-N-propargyl biotin, and trans-N-propargyl biotin and have examined these as substrates of biotin ligase from a number of species (Human, Saccharomyces cerevisiae, Bacillus subtilis, Pyrococcus horikoshii, Trypanosoma cruzi, Glardia lamblia, Methanococcus jannaschii and Escherichia coli (BirA) (Slavoff et al. J. Am. Chem. Soc., 2008, 130, 1160). It was demonstrated that the Saccharomyces cerevisiae enzyme could utilise cis-N-propargyl biotin whilst the Pyrococcus horikoshii enzyme could use desthiobiotin and cis-N-propargyl biotin as substrates. However, these analogues were not added to the 15-amino acid acceptor peptide with the same levels of efficiency as natural biotin was. Furthermore, due to the location of the bioorthogonal group on the biotin, it is anticipated that these biotin analogues would only have a low affinity for avidin, streptavidin, anti-biotin antibodies or other proteins that bind avidin. Once reacted with a suitable partner, these molecules are likely to exhibit even lower affinity for these proteins.

It is an aim of the present invention to provide novel biotin analogues that are substrates for biotin ligase and that are added to specific peptides, such as the Avitag™ peptide, with an acceptable level of efficiency, ideally similar to that of natural biotin.

A further aim of the present invention is to provide novel biotin analogues that have an acceptable, preferably reversible, binding affinity for avidin, streptavidin or other mutants or homologues thereof.

Yet a further aim of the present invention is to provide novel biotin analogues that are synthesised more readily with fewer steps and/or in a higher yield than biotin analogues prepared prior hereto.

Another aim of the present invention is to provide a method of labelling a target biomolecular structure, such as proteins and peptides, with a novel compound that may be used as either an affinity tag or as a specific point of covalent attachment for further molecular probes.

Accordingly, a first aspect of the present invention provides a biotin analogue comprising the ureido ring of natural biotin with at least one of a modified thiophene ring or a modified sidechain having a functional end group and at least one bio-orthogonally reactive chemical group located elsewhere in the side chain.

More preferably, the biotin analogue has the non-modified thiophene ring of natural biotin with only a modified sidechain having a functional end group and at least one bio-orthogonally reactive chemical group located elsewhere in the side chain. However, it is to be appreciated that the sulphur of the thiophene ring may be replaced with another group selected from CH₂, O, NH and C═O, for example if the functional groups in the modified valeryl side chain are unstable with the sulfur in the thiophene ring present. Alternatively, the analogue may comprise desthiobiotin with a modified valeryl sidechain.

Preferably, the functional end group is selected from the group consisting of a carboxylic acid, alcohol, aldehyde, amine, thiol and a halide.

The structure of natural biotin is as follows:

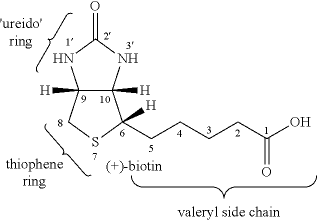

The numbering shown for the biotin backbone illustrated above is adhered to throughout this disclosure.

Preferably, at least one bio-orthogonally reactive group is selected from the group consisting of an azide, an alkyne, an alkene, a heterocyclic group, a diene group and/or one or more heteroatoms selected from S, N, Se, P and O. More preferably still, the reactive group is located on, or as part of, or in place of, the valeryl side chain of the biotin analogue, as represented by the following general formula:

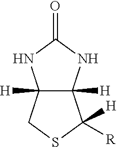

where R has a functional end group and includes at least one second functional group selected from the group consisting of an azide, an alkyne, an alkene, a heterocyclic ring, a diene and/or one or more heteroatoms selected from S, N, Se, P and O located elsewhere on the side chain. Preferably, the functional end group is selected from the group consisting of a carboxylic acid, amine, alcohol, thiol, aldehyde and a halide

The bio-orthoganally reactive group may be positioned at any one of positions 2 to 5 of the valeryl chain (—(CH₂)₄CO₂H). Preferably, the 5-carbon backbone of the valeryl sidechain is maintained in the biotin analogue according to the first aspect of the present invention. However, in an alternative embodiment, the valeryl group of the biotin analogue may contain a different number of carbon atoms, preferably between 1 to 10 carbon atoms, that may be SP, SP² or SP³ hybridised, and/or may include one or more heteroatoms selected from the group consisting of S, N, Se, P or O.

In one embodiment of the present invention, the bio-orthogonally reactive group is provided at position 2 of the valeryl side chain. A preferred biotin analogue according to a first aspect of the present invention is 2-azidobiotin, having an azide at position 2 of the valeryl side chain:

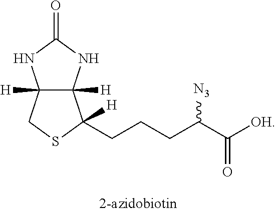

The R— and/or S-2-azidobiotin analogue may be used.

It is to be appreciated that the carboxylate end group may be substituted with a different functional group depending upon the intended application for the biotin analogue, as represented by the structures given below:

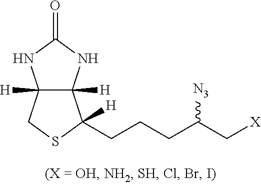

The end functional group may be further modified to form an intermediate that in vivo would be formed by an enzyme, such as Bir A, that may be used to attach the analogue to a target structure, such as a protein. Such groups are known in the art and include, for example, 5′-adenylate. Other modified end groups that may act as a substrate for BirA or other biotin ligases are carboxylates or activated esters. For example, the present invention includes 2-azidobiotin analogues of the following general formula:

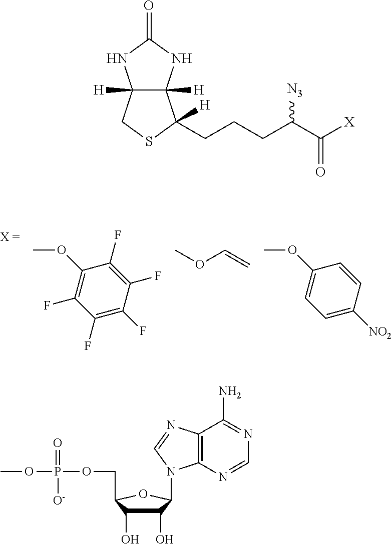

To this end, a further aspect of the present invention provides a biotin analogue according to the first aspect of the present invention having a modified end group selected from the group consisting of 5′-adenylate, related nucleotides and nucleotide analogues and simple activated esters, such as pentafluorophenyl, vinyl and p-nitrophenylesters. More preferably, the end group is a 5′-adenylate group. The modified analogue may be prepared by means of an enzyme or synthetically without the presence of an enzyme.

An example of such a preferred biotin analogue is 2-azidobiotinyl adenylate:

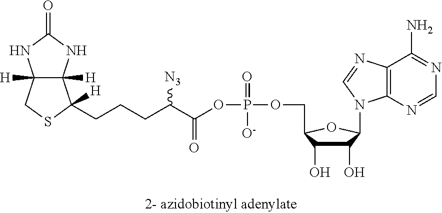

An alternative biotin analogue according to the first aspect of the present invention is 2-propargyl (2-propynyl) biotin, having an alkyne substituent at position 2 of the valeryl side chain:

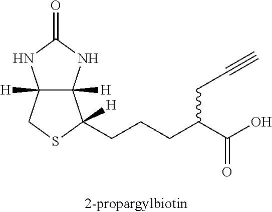

The alkyne substituent may be attached to the valeryl sidechain with a different number of carbon atoms, preferably being provided with 1 to 8 carbon atoms. The alkyne substituent may also be provided at a different position of the valeryl side chain. Again, the carboxylate end group could be replaced with another functional group, such as an amine, alcohol, thiol or halide.

Alternative biotin analogues according to the first aspect of the present invention may incorporate one of the following modified valeryl side chains:

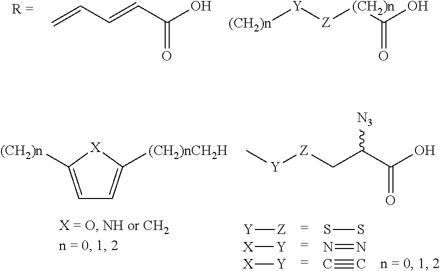

Yet further examples of biotin analogues according to the first aspect of the present invention include the following where X is CH₂, O, NH or C═O and Y is N, CH or S:

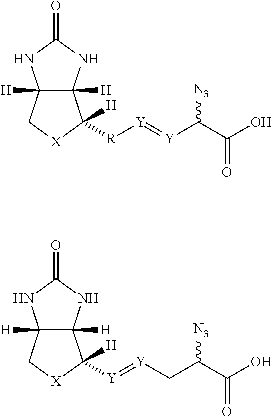

Any suitable method of synthesis may be used to prepare a biotin analogue according to a first aspect of the present invention. However, preferably, the biotin analogue is prepared from biotin. Preferably, the analogue 2-azidobiotin is prepared from biotin via an intermediate N,N′ di(p-methoxybenzyl)biotin methyl ester which is reacted with trisyl azide. Alternatively, an intermediate N,N′-dibenzylbiotin or its methyl ester may be used. In a preferred method of the present invention, the N,N′-dibenzylbiotin is subjected to saponification to yield N,N′-p-methoxybenzylbiotin which is highly soluble. The acid chloride of the methoxybenzylbiotin may then be formed by reaction with oxalyl chloride, followed by displacement with n-BuLi treated to oxazolidinone to form 3-(N,N′-p-methoxybenzylbiotinoyl) oxazolidin-2-one, followed by deprotection to form 2-azidobiotin.

A biotin analogue according to a first aspect of the present invention may be attached to specific target structure, such as proteins, peptides, luminescent, radioactive, MRI contrast agents, PET contrast agents, quantum dots or synthetic polymers. To this end, a second aspect of the present invention provides a biotin analogue according to a first aspect of the present invention attached to a specific target structure.

More preferably, the specific target structure is a biomolecular structure, especially a target protein or peptide to be labelled by the biotin analogue. The labelling may take place in vitro or in vivo. In a preferred embodiment of the invention, the target protein or peptide includes an acceptor peptide for acting as a specific substrate for an enzyme that will attach the target protein to the biotin analogue. The acceptor peptide may be fused to the target protein and/or biotin analogue either at the nucleic acid level or post-translationally. Generally, the enzyme will comprise biotin ligase from E. coli (BirA) or its mutants or homologues in other species.

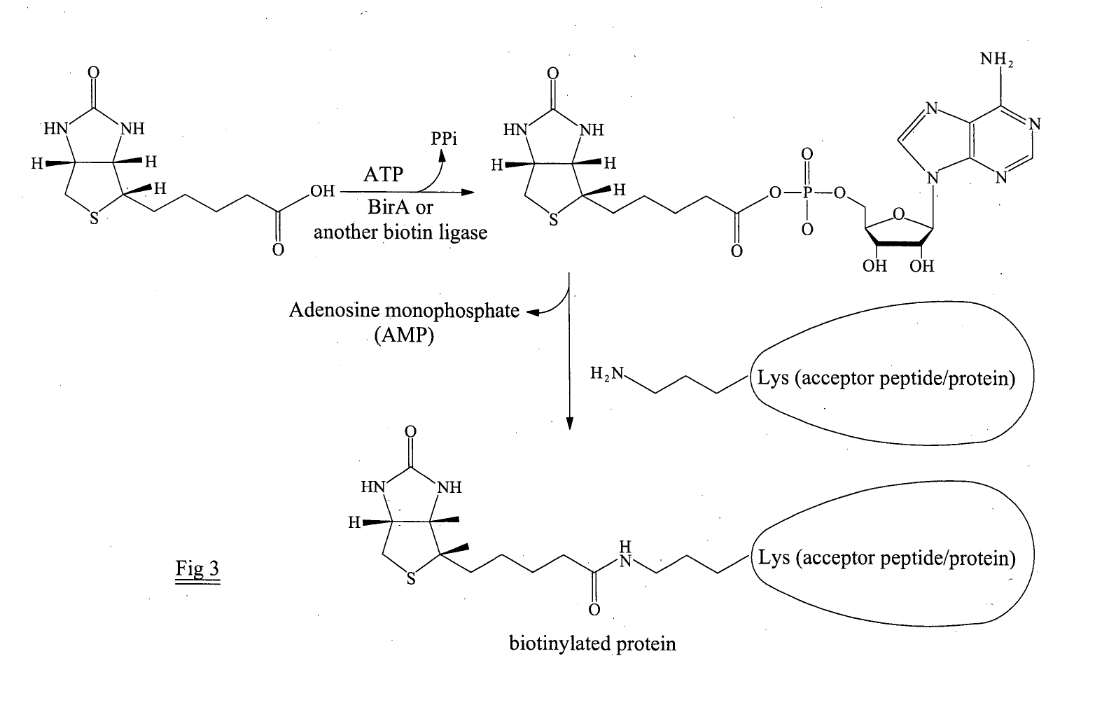

To this end, a third aspect of the present invention comprises a biotin analogue according to a first aspect of the present invention conjugated to a target biomolecular structure, preferably a protein, via an acceptor peptide. The method of attachment in vitro or in vivo generally comprises contacting the biotin analogue with a target protein (i.e. the protein to be labelled) that has been fused with an acceptor peptide (collectively described as the “fusion protein”) in the presence of biotin ligase and allowing for sufficient time for conjugation of the biotin analogue to the fusion protein. ATP must also be present. The scheme for the reaction is shown in FIG. 3 of the accompanying drawings. Times and reaction conditions suitable for biotin ligase activity will generally be comparable to those for wild type biotin ligase which are known in the art.

If the method is performed in vivo, the nucleic acid sequence encoding the fusion protein will be introduced into the cell and transcription and translation allowed to occur. If the method is performed in a cell free environment (in vitro), the fusion protein will simply be added to the reaction mixture (biotin analogue, BirA and ATP) in for example a test tube or a well of a multiwell plate.

As used herein, protein labelling in vivo means labelling of a protein in the context of a cell. The method can be used to label proteins that are intracellular proteins or cell surface proteins. The cell may be present in a subject (e.g., any organism, including an insect such as Drosophila, a rodent such as a mouse, a human, and the like) or it may be present in culture.

The biotin ligase may also be expressed by the cell in some instances. In other instances, however, the biotin ligase mutant may simply be added to the reaction mixture (if in vitro) or to the cell (if the target protein is a cell surface protein and the acceptor peptide is located on the extracellular domain of the target protein).

As will be appreciated from above, the acceptor peptide is preferably one that acts as a substrate for a biotin ligase or one of its mutants. The only known natural substrate in E. coli of wild type biotin ligase is lysine 122 of the biotin carboxyl carrier protein, BCCP. (Chapman-Smith et al. J. Nutr. 129:477 S-484S, 1999.) A 13-15 amino acid minimal substrate sequence encompassing lysine 122 has been identified as the minimal peptide recognition sequence for biotin ligase.

The reaction between biotin ligase and its substrate is referred to as orthogonal. This means that neither the ligase nor its substrate react with any other enzyme or molecule when present either in their native environment (i.e., a bacterial cell) or more importantly for the purposes of the invention in a non-native environment (e.g., a mammalian cell). Accordingly, the invention takes advantage of the high degree of specificity which has evolved between biotin ligase and its substrate.

As used herein, an “acceptor peptide” is a protein or peptide having an amino acid sequence that is accepted as a substrate for a biotin ligase, one of its mutants or homologues (i.e. a biotin ligase mutant recognizes and is capable of conjugating a biotin analogue or biotin to the peptide). The acceptor peptide may have an amino acid sequence of:

Leu Xaa.sub.1 Xaa.sub.2 Ile Xaa.sub.3 Xaa.sub.4 Xaa.sub.5 Xaa.sub.6 Lys Xaa.sub.7 Xaa.sub.8 Xaa.sub.9 Xaa.sub.10 (SEQ. ID NO:3), where Xaa.sub.1 is any amino acid, Xaa.sub.2 is any amino acid other than large hydrophobic amino acids (such as Leu, Val, Ile, Trp, Phe, Tyr); Xaa.sub.3 is Phe or Leu, Xaa4 is Glu or Asp; Xaa.sub.5 is Ala, Gly, Ser, or Thr; Xaa.sub.6 is Gln or Met; Xaa.sub.7 is Ile, Met, or Val; Xaa.sub.8 is Glu, Leu, Val, Tyr, or Ile; Xaa.sub.9 is Trp, Tyr, Val, Phe, Leu, or Ile; and Xaa.sub.10 is preferably Arg or His but may be any amino acid other than acidic amino acids such as Asp or Glu.

In a preferred embodiment, the acceptor peptide comprises one of the following amino acid sequences where the point of attachment is the lysine residue in bold:

SEQ. ID No. 4
GLNDIFEAQKEWHE
SEQ. ID No. 5
DTLCIVEAMKMMNQI
SEQ. ID No. 6
GLNDIFEAQKIEWHE

In other embodiments, the acceptor peptide comprises other amino acid sequences which are known, or subsequently become known, to act as a subtrate for biotin ligase or one of its mutants. Examples are described in U.S. Pat. Nos. 5,723,584; 5,874,239 and 5,932,433, the entire contents of which are herein incorporated by reference.

Acceptor peptides can be synthesized using standard peptide synthesis techniques or fused in their DNA encoded form to the gene of interest using standard molecular biology techniques. They are also commercially available, for example under the trade name BioEase™ from Invitrogen and under the trade name AviTag™ from Avidity (Boulder, Colo.)—see SEQ. ID No. 6 above. SEQ ID No. 6 is incorporated into proteins at the N- or C-terminals using Avitag™ technology in the following forms:

| | |
|---|---|
| | (SEQ.ID No. 19) |
| N-terminal tag sequence: | MSGLNDI FEAQK I EWHE |
| | (SEQ.ID No. 20) |
| C-terminal tag sequence | LERAP GGLNDI FEAQK I EWHE |

Other examples of known acceptor peptide sequences for a variety of organisms are given below (respectively SEQ. ID No.s 7 to 18 in descending order):

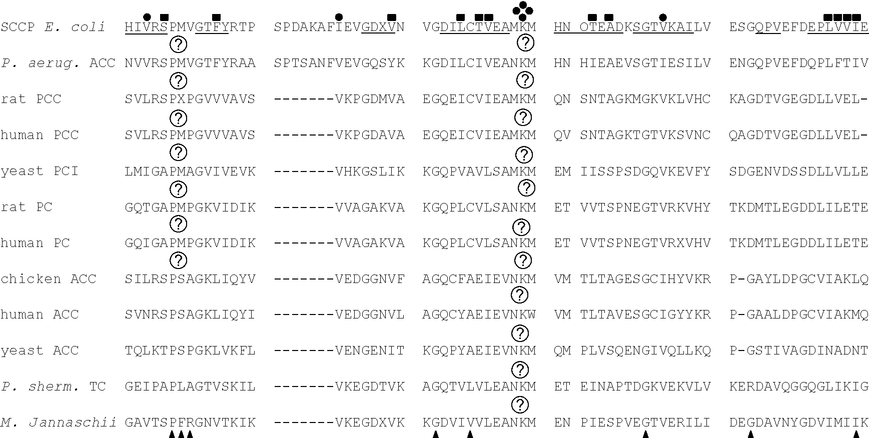

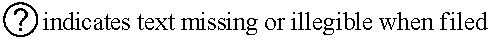

Residues forming β-strands in the 3-dimensional structure of Escherichia coli biotin carboxyl carrier protein (BCCP) are underlined; hydrophobic core residues are indicated by ▪. The biotinylated lysine residue is marked D. Shading indicates residues very highly conserved in all biotin domains for which sequence data are available. Positions at which amino acid substitution is known to reduce the efficiency of biotinylation are indicated by ▴(Alignment was done using Clustal W (reproduced from A. Chapman-Smith and J. E. Cronan J. Nutritional Science 1999, 129 477S).

The acceptor peptide is used in the methods of the invention to tag target proteins that are to be labelled by biotin ligase. The acceptor peptide and target protein may be fused to each other either at the nucleic acid or amino acid level. Recombinant DNA technology for generating fusion nucleic acids that encode both the target protein and the acceptor peptide are known in the art. Additionally, the acceptor peptide may be fused to the target protein post-translationally, for example through native chemical ligation. Such linkages may include cleavable linkers or bonds which can be cleaved once the desired labeling is achieved. Preferably, the valeryl side chain is modified to incorporate the cleavable linker between the bicyclic core of the biotin analogue and the terminal carboxylate and bioorthogonal group. Once cleaved, the carboxylate group and adjacent carbons bearing the bioorthogonal reactive group on the acceptor peptide are left ready for further reaction but the acceptor peptide/protein is no longer able to be bound by avidin/streptavidin or their homologues. Such bonds may be cleaved by exposure to a particular pH, or energy of a certain wavelength, and the like. Examples of the types of functionality that could be cleaved include but are not restricted to disulfide bonds (—S—S—), imines (—C═N—) and diazo (—N═N—) compounds.

The acceptor peptide can be fused to the target protein at any position. In some instances, it is preferred that the fusion not interfere with the activity of the target protein, in which case the acceptor peptide is fused to the protein at positions that do not interfere with the activity of the protein.

Generally, the acceptor peptides can be C- or N-terminally fused to the target proteins. In still other instances, it is possible that the acceptor peptide is fused to the target protein at an internal position (e.g., a flexible internal loop). These proteins are then susceptible to specific tagging by biotin ligase and biotin ligase mutants in vivo and in vitro.

Preferably, the biotin analogue of the present invention is able to bind with avidin, streptavidin or their homologues, or anti-biotin antibodies. The biotin analogue may form very high or moderate affinity non-covalent interactions with the aforementioned substrates. Such interactions may form with the biotin analogue itseld and/or with the target protein and/or label attached thereto.

It is preferable for the biotin analogue (with or without the target protein and/or label attached thereto) to be releasable from its interaction with avidin, streptavidin or their mutants or homologues or from its interaction with anti-biotin antibody by a change in conditions, such as a change in pH, salt concentration or by the addition of biotin or another or its analogues. For example, strep-tag II peptide sequence (Trp-Ser-His-Pro-Gln-Phe-Glu-Lys—SEQ. ID No. 21)(Schmidt TGM and Skerra A, NATURE PROT. 2007, 2, 1528-1535) or other molecules known to have a moderate or good binding affinity for avidin, streptavidin, their mutants or homologues or an anti-biotin antibody such as 4-hydroxyazobenzene-2-carboxylic acid (HABA).

Once attached to the acceptor peptide/protein, the biotin analogue according to the first aspect of the invention may be used as an affinity tag/ligand to allow the biotinylated protein to be separated from non-biotinylated proteins in a mixture by binding it to avidin, streptavidin or their mutants and homologues, or to anti-biotin antibodies that have been immobilised on a surface, polymer or (magnetic) bead support. Alternatively or additionally, the bio-orthogonal functionality in the valeryl side chain can be selectively reacted with either an alkyne (Huisgen cyclisation) or Phosphine (Staudinger ligation including traceless variations)(Baskin and Bertozzi, QSAR & Comb. Sci. 2007, 26, 1211-1219; Hackenberger and Schwarzer Angew. Chem. Int. Ed. 2008, 47, 10030-10074) to allow the acceptor peptide/protein to either be functionalised with a chemical label, a second protein or other biopolymer, or be attached to a synthetic polymer (such as a dendrimer) or a surface.

The attachment to the acceptor protein, binding to avidin-/streptavidin and bioorthogonal chemistry described above may be conducted in any order.

The aforementioned method of conjugation of the biotin analogue to the fusion protein is independent of the protein type and thus any protein can be labelled in this manner. The product of this labelling reaction may or may not be directly detectable, depending upon the nature of the biotin analogue. Accordingly, it may be necessary to react the conjugated biotin analogue with a detectable label. If the method is performed in vivo, the detectable label is preferably one capable of diffusion into a cell. If the biotin analogue is too polar to cross the cell membrane, the analogue should be derivatised to a less polar form, for example to their ester form (including but not limited to methyl, ethyl or pivaloyl esters). If the method is used to label a cell surface protein, then preferably the biotin analogue is labelled with a membrane impermeant label in order to reduce entry and accumulation of the label intracellularly. The biotin analogue may be labelled prior to or after conjugation to the fusion protein.

Labelling of proteins allows one to track the movement and activity of such proteins. It also allows cells expressing such proteins to be tracked and imaged, as the case may be. The methods can be used in cells from virtually any organism including insect, yeast, frog, worm, fish, rodent, human and the like.

The method can be used to label virtually any protein. Examples include but are not limited to signal transduction proteins (e.g., cell surface receptors, kinases, adapter proteins), nuclear proteins (transcription factors, histones), mitochondrial proteins (cytochromes, transcription factors) and hormone receptors.

As mentioned above, the biotin analogue according to the first aspect of the present invention may be directly detectable or indirectly detectable. The biotin analogue may be directly detectable either through binding to suitably functionalised avidin, streptavidin or their homologues or a labelled anti-biotin antibody.

Alternatively or additionally, the biotin analogue may undergo reaction with another detectable moiety (before or after conjugation to the acceptor peptide) by means of a bioorthogonal ligation reaction for coupling the analogue to a detectable moiety, such as a fluorophore. The resulting moiety may be a hydrazine, phosphine or azide but is not so limited. To this end, a fourth aspect of the present invention provides a biotin analogue according to a first aspect of the present invention coupled to a directly or indirectly detectable label.

Accordingly, biotin analogues that are not themselves directly detectable must be reacted with a detectable moiety. Each biotin analogue in this category will undergo a specific reaction dependent upon its functional groups and that of its reaction partner. For example, azides may be reacted with phosphines in a Staudinger reaction. Azides and aryl phosphines generally have no cellular counterparts. As a result, the reaction is quite specific. Azide variants with improved stability against hydrolysis in water at pH 6-8 are also useful in the methods of the invention. The alkyne/azide [3+2] cycloaddition chemistry, based on Click chemistry (Wang et al. J. Am. Chem. Soc. 125:11164-11165, 2003), is also specific, in particular when the two reactive partners do not have cellular counterparts (i.e., the two functional groups are non-naturally occurring).

The biotin analogues can also be fluorogenic. As used herein, a fluorogenic compound is one that is not detectable (e.g., fluorescent) by itself, but when conjugated to another moiety becomes fluorescent. An example of this is non-fluorescent coumarin phosphine which reacts with azides to produce fluorescent coumarin.

As stated above, the biotin analogues can be conjugated to detectable labels. A “detectable label” as used herein is a molecule or compound that can be detected by a variety of methods including fluorescence, electrical conductivity, radioactivity, size, and the like. The label may be of a chemical (e.g., carbohydrate, lipid, etc.), peptide or nucleic acid nature although it is not so limited. The label can be detected directly for example by its ability to emit and/or absorb light of a particular wavelength. A label can be detected indirectly by its ability to bind, recruit and, in some cases, cleave (or be cleaved by) another compound, thereby emitting or absorbing energy. An example of indirect detection is the use of an enzyme label which cleaves a substrate into visible products.

The type of label used will depend on a variety of factors, such as but not limited to the nature of the protein ultimately being labelled. The label should be sterically and chemically compatible with the biotin analogue, the acceptor peptide and the target protein. In most instances, the label should not interfere with the activity of the target protein.

A wide variety of labelling agents exist which may be selected as appropriate for providing suitable detection of the biotin analogue and its conjugated protein. Generally, the label can be selected from the group consisting of a fluorescent molecule, a chemiluminescent molecule (e.g., chemiluminescent substrates), a phosphorescent molecule, a radioisotope, an enzyme, an enzyme substrate, an affinity molecule, a ligand, an antigen, a hapten, an antibody, an antibody fragment, a chromogenic substrate, a contrast agent, an MRI contrast agent, a PET label, a phosphorescent label, and the like.

Specific examples of suitable labels include the following: radioactive isotopes such as ³²P or ³H; haptens such as digoxigenin and dinitrophenyl; affinity tags such as a FLAG tag, an HA tag, a histidine tag, a GST tag; enzyme tags such as alkaline phosphatase, horseradish peroxidase, beta-galactosidase, etc.

Other labels include fluorophores such as, for example, fluorescein isothiocyanate (“FITC”), tetramethylrhodamine isothiocyanate (“TRITC”) and 4,4-difluoro-4-bora-3a, and 4a-diaza-s-indacene (“BODIPY”).

The labels can also be antibodies or antibody fragments or their corresponding antigen, epitope or hapten binding partners. Detection of such bound antibodies and proteins or peptides is accomplished by techniques well known to those skilled in the art and thus need not be described in detail herein. Antibody/antigen complexes which form in response to hapten conjugates are easily detected by linking a label to the hapten or to antibodies which recognize the hapten and then observing the site of the label. Alternatively, the antibodies can be visualized using secondary antibodies or fragments thereof that are specific for the primary antibody used.

Polyclonal and monoclonal antibodies may also be used. Antibody fragments include Fab, F(ab).sub.2, Fd and antibody fragments which include a CDR3 region. The conjugates can also be labeled using dual specificity antibodies.

Alternatively, the label may be a contrast agent. Contrast agents are molecules that are administered to a subject to enhance a particular imaging modality such as but not limited to X-ray, ultrasound, and MRI. Suitable contrast agents are known in the art and need not be further described herein.

The label may be a positron emission tomography (PET) label such as 99 m technetium or 18FDG.

The label may also be a singlet oxygen radical generator including a porphyrin or other group previously used in photodynamic therapy, such as (but not limited to) resorufin, malachite green, fluorescein, benzidine and its analogues. These molecules are useful in EM staining and can also be used to induce localized toxicity.

The label may also be an analyte-binding group such as but not limited to a metal chelator (e.g., a copper chelator). Examples of metal chelators include EDTA, EGTA, and molecules having pyridinium substituents, imidazole substituents, and/or thiol substituents. These labels can be used to analyze local environment of the target protein (e.g., Ca.sup.2+concentration).

The label may comprise a heavy atom carrier. Examples of a heavy atom carrier are iodine, iron or gadolinium. Such labels are particularly useful for X-ray crystallographic study of the target protein. Heavy atoms used in X-ray crystallography include but are not limited to Au, Pt and Hg.

The label may also be a photoactivatable cross-linker. A photoactivable cross linker is a cross linker that becomes reactive following exposure to radiation (e.g., a ultraviolet radiation, visible light, etc.) such as those selected from the group consisting of benzophenones, aziridines, diazirines and trifluoromethyketones and which are known in the art.

The label may also be a photoswitchable label. A photoswitch label is a molecule that undergoes a conformational change in response to radiation. For example, the molecule may change its conformation from cis to trans and back again in response to radiation. The wavelength required to induce the conformational switch will depend upon the particular photoswitch label. Examples of photoswitchable labels include azobenzene, 3-nitro-2-naphthalenemethanol and spyropyrans.

The label may also be a photolabile protecting group. Examples of photolabile protecting group include a nitrobenzyl group, a dimethoxy nitrobenzyl group, nitroveratryloxycarbonyl (NVOC), 2-(dimethylamino)-5-nitrophenyl (DANP), Bis(o-nitrophenyl)ethanediol, brominated hydroxyquinoline, and coumarin-4-ylmethyl derivative. Photolabile protecting groups are useful for photocaging reactive functional groups.

The label may comprise non-naturally occurring amino acids. Modifications of cysteines, histidines, lysines, arginines, tyrosines, glutamines, asparagines, prolines, and carboxyl groups are known in the art and are described in U.S. Pat. No. 6,037,134. These types of labels can be used to study enzyme structure and function.

The label may be an enzyme or an enzyme substrate. Examples of these include (enzyme (substrate)): Alkaline Phosphatase (4-Methylumbelliferyl phosphate Disodium salt; 3-Phenylumbelliferyl phosphate Hemipyridine salt); Aminopeptidase (L-Alanine-4-methyl-7-coumar-inylamide trifluoroacetate; Z-L-arginine-4-methyl-7-coumarinylamide hydrochloride; Z-glycyl-L-proline-4-methyl-7-coumarinylamide); Aminopeptidase B (L-Leucine-4-methyl-7-coumarinylamide hydrochloride); Aminopeptidase M (L-Phenylalanine 4-methyl-7-coumarinylamide trifluoroacetate); Butyrate esterase (4-Methylumbelliferyl butyrate); Cellulase (2-Chloro-4-nitrophenyl-beta-D-cellobioside); Cholinesterase (7-Acetoxy-1-methylquinolinium iodide; Resorufin butyrate); alpha-Chymotrypsin, (Glutaryl-L-phenylalanine 4-methyl-7-coumarinylamide)-; N—(N-Glutaryl-L-phenylalanyl)-2-aminoacridone; N—(N-Succinyl-L-phenylala-nyl)-2-aminoacridone); Cytochrome P450 2B6 (7-Ethoxycoumarin); Cytosolic Aldehyde Dehydrogenase (Esterase Activity) (Resorufin acetate); Dealkylase (O.sup.7-Pentylresorufin); Dopamine beta-hydroxylase (Tyramine); Esterase (8-Acetoxypyrene-1,3,6-trisulfonic acid Trisodium salt; 3-(2 Benzoxazolyl)umbelliferyl acetate; 8-Butyryloxypyrene-1,3,6-tr-isulfonicacid Trisodium salt; 2′,7′-Dichlorofluorescin diacetate; Fluorescein dibutyrate; Fluorescein dilaurate; 4-Methylumbelliferyl acetate; 4-Methylumbelliferyl butyrate; 8-Octanoyloxypyrene-1,3,6-trisulf-onic acid Trisodium salt; 8-Oleoyloxypyrene-1,3,6-trisulfonic acid Trisodium salt; Resorufin acetate); Factor X Activated (Xa) (4-Methylumbelliferyl 4-guanidinobenzoate hydrochloride Monohydrate); Fucosidase, alpha-L-(4-Methylumbelliferyl-alpha-L-fucopyranoside); Galactosidase, alpha-(4-Methylumbelliferyl-alpha-D galactopyranoside); Galactosidase, beta-(6,8-Difluoro-4-methylumbelliferyl-beta-D-galactopyr-anoside; Fluorescein di(beta-D-galactopyranoside); 4-Methylumbelliferyl-al-pha-D-galactopyranoside; 4-Methylumbelliferyl-beta-D-lactoside: Resorufin-beta-D-galactopyranoside; 4-(Trifluoromethyl)umbelliferyl-beta-D-galactopyranoside; 2-Chloro-4-nitrophenyl-beta-D-lactoside); Glucosaminidase, N-acetyl-beta-(4-Methylumbelliferyl-N-acetyl-beta-D-glu-cosaminide Dihydrate); Glucosidase, alpha-(4-Methylumbelliferyl-alpha-D-gl-ucopyranoside); Glucosidase, beta-(2-Chloro-4-nitrophenyl-beta-D-glucopyr-anoside; 6,8-Difluoro-4-methylumbelliferyl-beta-D-glucopyranoside; 4-Methylumbelliferyl-beta-D-glucopyranoside; Resorufin-beta-D-glucopyrano-side; 4-(Trifluoromethyl)umbelliferyl-beta-D-glucopyranoside); Glucuronidase, beta-(6,8-Difluoro-4-methylumbelliferyl-beta-D-glucuronide Lithium salt; 4-Methylumbelliferyl-beta-D-glucuronide Trihydrate); Leucine aminopeptidase(L-Leucine-4-methyl-7-coumarinylamide hydrochloride); Lipase (Fluorescein dibutyrate; Fluorescein dilaurate; 4-Methylumbelliferyl butyrate; 4-Methylumbelliferyl enanthate; 4-Methylumbelliferyl oleate; 4-Methylumbelliferyl palmitate; Resorufin butyrate); Lysozyme (4-Methylumbelliferyl-N,N′,N′-triacetyl-beta-chitotri-oside); Mannosidase, alpha-(4-Methylumbelliferyl-alpha-D-mannopyranoside-); Monoamine oxidase (Tyramine); Monooxygenase (7-Ethoxycoumarin); Neuraminidase (4-Methylumbelliferyl-N-acetyl-alpha-D-neuraminic acid Sodium salt Dihydrate); Papain (Z-L-arginine-4-methyl-7-coumarinylamide hydrochloride); Peroxidase (Dihydrorhodamine 123); Phosphodiesterase (1-Naphthyl 4-phenylazophenyl phosphate; 2-Naphthyl 4-phenylazophenyl phosphate); Prolyl endopeptidase (Z-glycyl-L-proline-4-methyl-7-coumariny-lamide; Z-glycyl-L-proline-2-naphthylamide; Z-glycyl-L-proline-4-nitroanil-ide); Sulfatase (4-Methylumbelliferyl sulfate Potassium salt); Thrombin (4-Methylumbelliferyl 4-guanidinobenzoate hydrochloride Monohydrate); Trypsin (Z-L-arginine-4-methyl-7-coumarinylamide hydrochloride; 4-Methylumbelliferyl 4-guanidinobenzoate hydrochloride Monohydrate); Tyramine dehydrogenase (Tyramine).

It is to be understood that many of the foregoing labels can also be biotin analogues. That is, depending upon the particular biotin ligase used, the various afore-mentioned labels may function as biotin analogues. As such, these biotin analogues would be considered to be directly detectable biotin analogues. In some cases, they would not require further modification.

The labels can be attached to the biotin analogues either before or after the analogue has been conjugated to the acceptor peptide, presuming that the label does not interfere with the activity of biotin ligase. Labels can be attached to the biotin analogs by any mechanism known in the art.

The labels attached to the biotin analogue conjugate may be detected using an appropriate detection system for the label concerned. The detection system is selected from any number of detection systems known in the art and thus need not be discussed in any further detail herein. The detection system may comprise, for example, a fluorescent detection system, a photographic film

detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system.

The labelling methods of the invention generally rely on the activity of wild type biotin ligase or mutants that recognize and conjugate biotin analogues onto fusion proteins via the acceptor peptide. The invention provides biotin ligase wild type and mutants that recognize'biotin analogues, and in some instances, biotin itself. As used herein, a biotin ligase mutant is a variant of biotin ligase that is enzymatically active towards a biotin analogue (such as those described herein). As used herein, “enzymatically active” means that the mutant is able to recognize and conjugate biotin or a biotin analogue to the acceptor peptide.

The biotin ligase mutant can have various mutations, including addition, deletion or substitution of one or more amino acids.

The biotin ligase mutant may retain some level of activity for biotin. Its binding affinity for biotin may be similar to that of wild type biotin ligase. Preferably, the mutant has higher binding affinity for a biotin analogue than it does for biotin. Consequently, biotin conjugation to an acceptor peptide would be lower in the presence of a biotin analogue. In still other embodiments, the biotin ligase mutant has no binding affinity for biotin.

Biotin ligase mutants can be made using standard molecular biology techniques known to those of ordinary skill in the art. For example, the mutants may be formed by transcription and translation from a nucleic acid sequence encoding the mutant. Such nucleic acid sequences can be made based on the teaching of wild type biotin ligase sequence and the position and type of amino acid substitution.

Codon optimised biotin ligase may be used for the formation of the conjugated protein. Codon optimisation of the Bir A enzyme leads to a higher expression of the protein and an improved efficiency of biotinylation of target proteins. (Cristele Gilbert et al., Journal of Biotechnology. Vol 116, Issue 3, 30 Mar. 2005, pages 245-249).

The biotin analogue binds to a biotin ligase in the interaction and activation domain. Preferably it binds with an affinity comparable to the binding affinity of wild type biotin ligase to biotin. However, biotin analogues that bind with lower affinities are still useful according to the invention. In some important embodiments, the biotin analogue is not recognized by wild type biotin ligase derived from either E. coli or from other cell types (e.g., the cell in which the labelling reaction is proceeding).

The biotin analogue may be labelled with a compound that prevents it from crossing cell membranes. Alternatively, depending upon its intended application, the biotin analogue may be labelled with a compound that improves the rate it can cross bacterial, fungal, plant, mammalian or other eukaryotic membranes.

The invention provides in some instances biotin ligases and/or biotin analogues in an isolated form. As used herein, an isolated biotin ligase is a biotin ligase that is separated from its native environment in sufficiently pure form so that it can be manipulated or used for any one of the purposes of the invention. Thus, isolated means sufficiently pure to be used (i) to raise and/or isolate antibodies, (ii) as a reagent in an assay, or (iii) for sequencing, etc.

Isolated biotin analogues similarly are analogues that have been substantially separated from either their native environment (if it exists in nature) or their synthesis environment. Accordingly, the biotin analogues are substantially separated from any or all reagents present in their synthesis reaction that would be toxic or otherwise detrimental to the target protein, the acceptor peptide, the biotin ligase, or the labelling reaction.

Various methods of the invention also require expression of fusion proteins in vivo. The fusion proteins are generally recombinantly produced proteins that comprise the biotin ligase acceptor peptides. Such fusions can be made from virtually any protein and those of ordinary skill in the art will be familiar with such methods. Further conjugation methodology is also provided in U.S. Pat. Nos. 5,932,433; 5,874,239 and 5,723,584.

In some instances, it may be desirable to place the biotin ligase and possibly the fusion protein under the control of an inducible promoter. An inducible promoter is one that is active in the presence (or absence) of a particular moiety. Accordingly, it is not constitutively active. Examples of inducible promoters are known in the art and include the tetracycline responsive promoters and regulatory sequences such as tetracycline-inducible T7 promoter system, and hypoxia inducible systems (Hu et al. Mol Cell Biol. 2003 December; 23(24):9361-74). Other mechanisms for controlling expression from a particular locus include the use of synthetic short interfering RNAs (siRNAs).

The components, as described above, are administered in effective amounts for labelling of the target structure. The effective amount will depend upon the mode of administration, the location of the cells being targeted, the amount of target structure present and the level of labelling desired.

It is to be appreciated that the biotin analogues according to the invention may be used in a wide variety of applications. For example, the analogues may be used in processes including, but not limited to, protein purification; cell sorting; in vivo protein trafficking; protein immobilisation; protein detection; multiprotein assemble.

The invention may also be used to provide key components of biosensors; diagnostic kits; drug delivery; drug targeting; drug activation systems; high throughput assays; proximity assays(including those involving resonance energy transfer); binding affinity assays as well as other assays and devices.

It is to be appreciated that the invention is not limited to attachment of the biotin analogue to a label or target structure by means of the enzyme Bir A or other enzymes with biotin ligase activity. Standard coupling chemistry may be employed (i.e. without the involvment of enzymes) to attach the analogue to certain biomolecular structures, such as DNA and RNA, proteins, polysaccharides, glycoproteins etc. For example, standard peptide coupling chemistry could be utilised (EDC, DCC, pyBOP or other carboxylate activating agents). Other methods include, but are not limited to, those where biotin is similarly used i.e. methods of biotinylation. These include, but are not limited, primary amine, sulfhydryl, carboxyl, glycoprotein and non-specific biotinylation. It is to be appreciated that biotin analogues according to the first aspect of the present invention having the valeryl side chain carboxylate could also be substituted by an amine, alcohol, thiol, aldehyde or halide so that it can be reacted with suitable nucleophilic or electrophilic partners to form a new covalent bond. The biotin analogue may also be attached to a protein or synthetic polymer through the formation of a metallic complex.

2-Azidobiotin can be further modified via the azido functional group pre- or post attachment to one of the above species. Modification of other analogues would similarly be pre or post attachment. Modifications include, but are not limited to, all those listed herein in relation to attachment of the 2-azidobiotin and biotin analogues to proteins using the BirA/acceptor protein method described. These chemically modified species can be used in the processes described above, but their use is not here limited. Additional uses include, but are not limited to, array technologies, ELISA, point-of-care diagnostics, biological imaging, lab-on-a-chip technologies and technologies which utilise biotin-(strep)avidin binding.

The invention will be more fully understood by reference to the following examples in which Examples 1A and 1B describe one synthetic process for the synthesis of 2-Azidobiotin, a novel biotin analogue according to one embodiment of the first aspect of the present invention and investigate optimisation of the process; Example 2 describes an alternative route for the synthesis of 2-Azidobiotin; Example 3 describes a further route for the synthesis of 2-Azidobiotin according to a preferred method of the invention; Example 4 investigates the addition of 2-Azidobiotin to an acceptor peptide using Biotin ligase; Example 5 studies the binding affinity of 2-Azidobiotin for Avidin; Example 6 investigates the reaction of 2-Azidobiotin with a bioorthogonally functionalised tag and Example 7 investigates the binding affinity of two biotin analogues that fall outside the scope of the invention and compares this with that of 2-Azidobiotin, and with reference to the accompanying drawings, in which

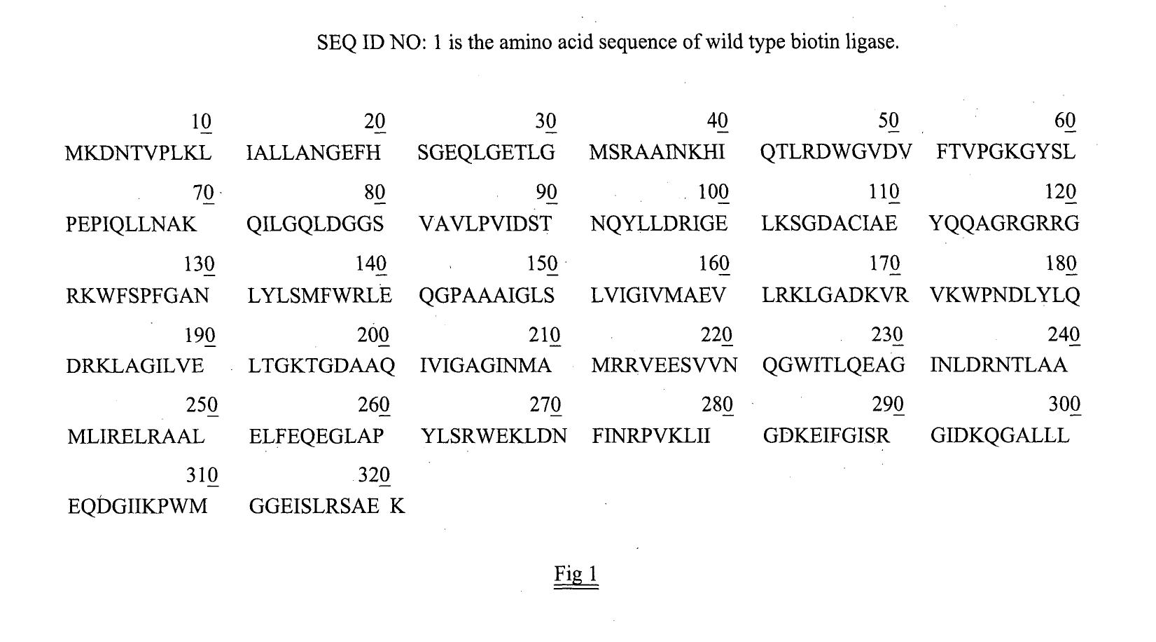

FIG. 1 is the amino acid sequence of wild type biotin ligase (SEQ.ID No. 1);

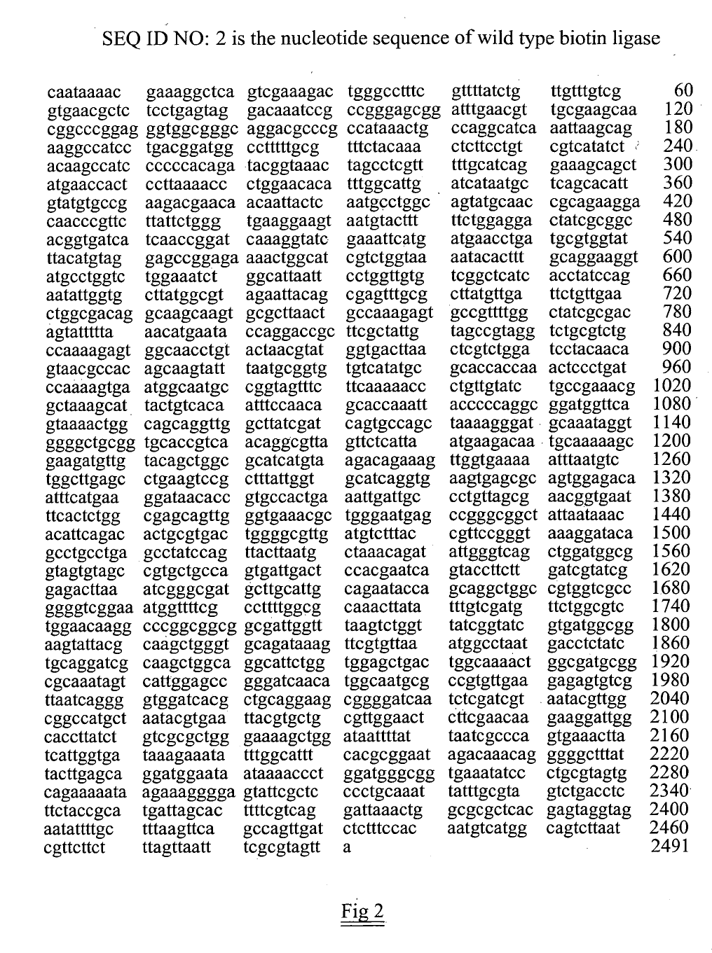

FIG. 2 is the nucleotide sequence of wild type biotin ligase (SEQ. ID No. 2);

FIG. 3 illustrates the biotinylation of the lysine side chain of an acceptor peptide sequence of a protein catalysed by biotin ligase (BirA);

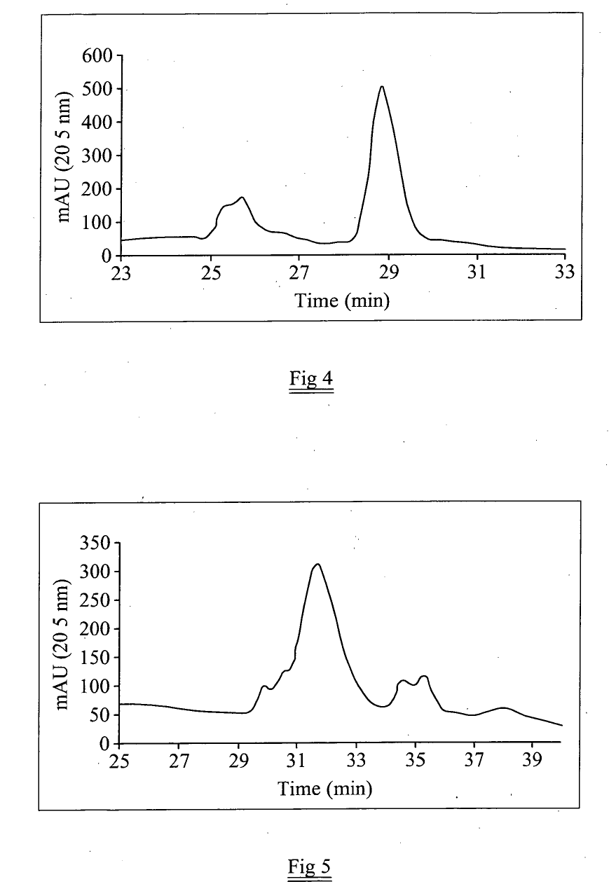

FIG. 4 is a HPLC trace of 2-azidobiotin-acceptor adduct attached using BirA;

FIG. 5 is a HPLC trace of biotin attached to the same peptide as that attached to the 2-azidobiotin of FIG. 4 using BirA;

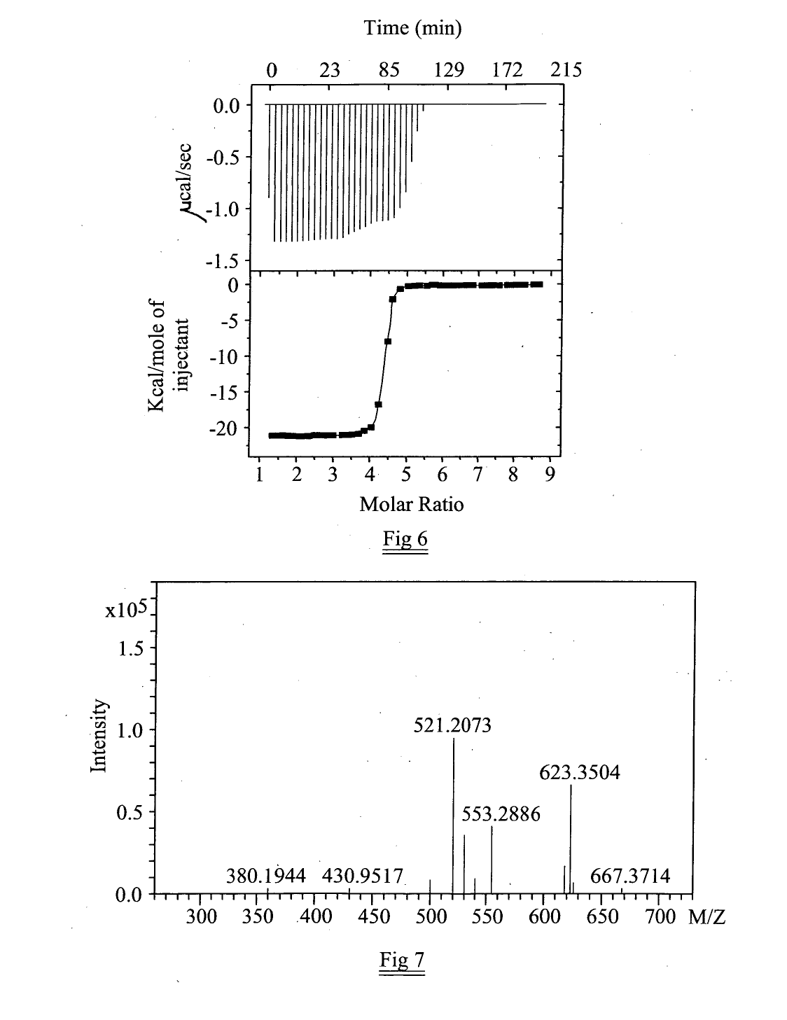

FIG. 6 shows the isothermal titration calorimetry data for 2-azidobiotin with avidin;

FIG. 7 is a spectrum for the biotin analogue linked to a coumarin derivative through a triazole formed in a Huisgen cycloaddition reaction;

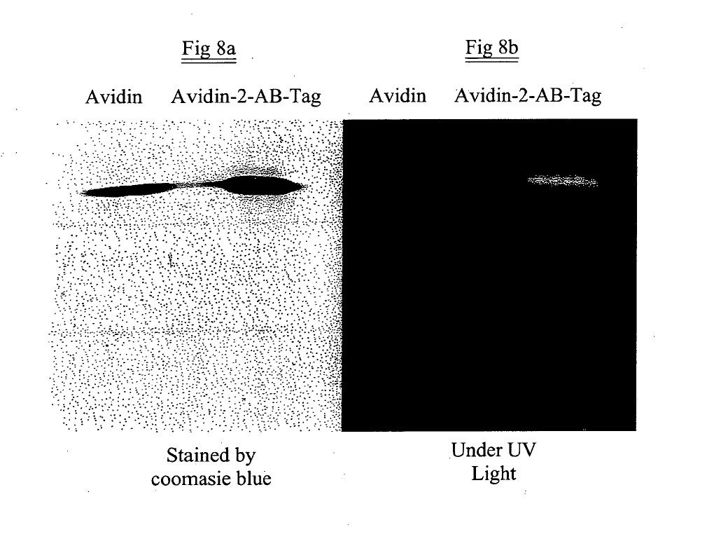

FIGS. 8a and 8b illustrate respectively native gels stained with coomasie blue and observed under UV-light to demonstrate binding of 2-azidobiotin to avidin following click chemistry attachment of a fluorophore; and

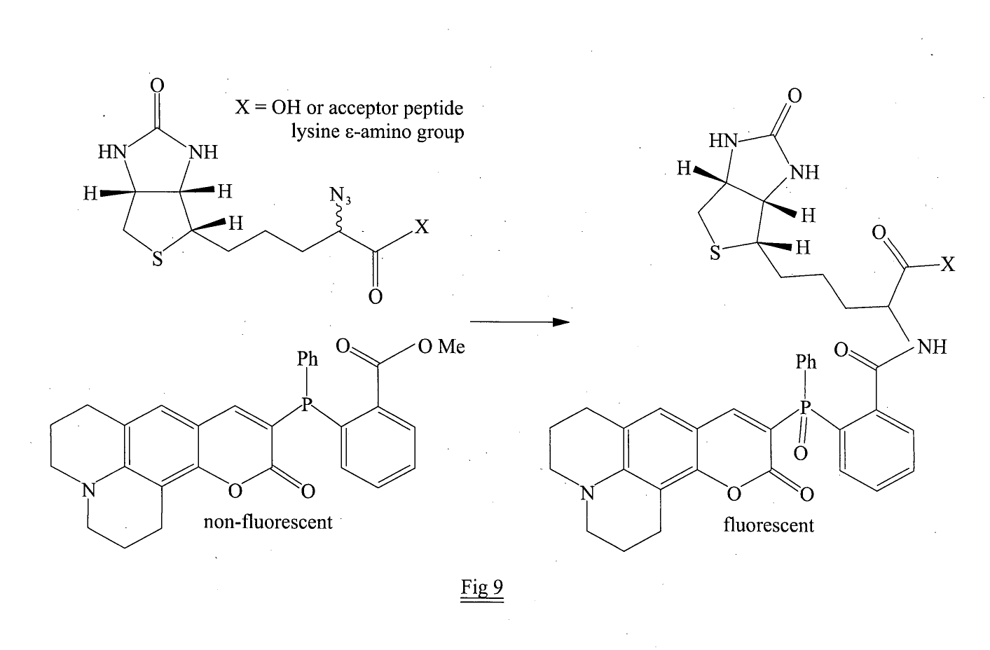

FIG. 9 shows the Staudinger-Bertozzi reaction between 2-azidobiotin and a fluorogenic dye that is activated by the Staudinger ligation (G. A. Lemieux, C. L. de Graffenried and C. R. Bertozzi, J. Am. Chem. Soc. 2003, 125, 4708-4709).

The present invention is concerned with the design, synthesis and applications of novel biotin analalogues that may be substrates of the biotin ligase from E. coli or its mutants, or homologues of this enzyme found in other species. Such analogues are bound with moderate to high affinity by the proteins avidin, streptavidin (or their homologues) or anti-biotin antibodies or synthetic equivalents. The biotin analogues are functionalised with chemically reactive groups that are capable of undergoing highly selective (bio-orthogonal) chemical reactions when in the presence of complex media such as biological fluids or the cytoplasm of a cell to form stable bonds with specific reaction partners, such as proteins.

In particular, the novel biotin analogues described herein have a number of key properties that have not been observed with biotin analogues previously disclosed. Firstly, the analogues according to the invention act as a substrate for BirA biotin ligase and are added to the Avitag™ peptide (GLNDIFEAQKIEWHE*⁻SEQ. ID No. 22) in the presence of ATP with a similar efficiency to natural biotin. The biotin analogue has also been shown to have reasonable (K_{d}˜10⁻⁷ M) binding affinity for avidin using isothermal titration calorimetry and has significantly higher affinity for avidin than the azidobiotin analague reported by Slavoff et al. vide supra.

### Example 1A

### Synthesis of 2-Azidobiotin

2-Azidobiotin was prepared in 5 steps from biotin and in 12% overall yield as given below.

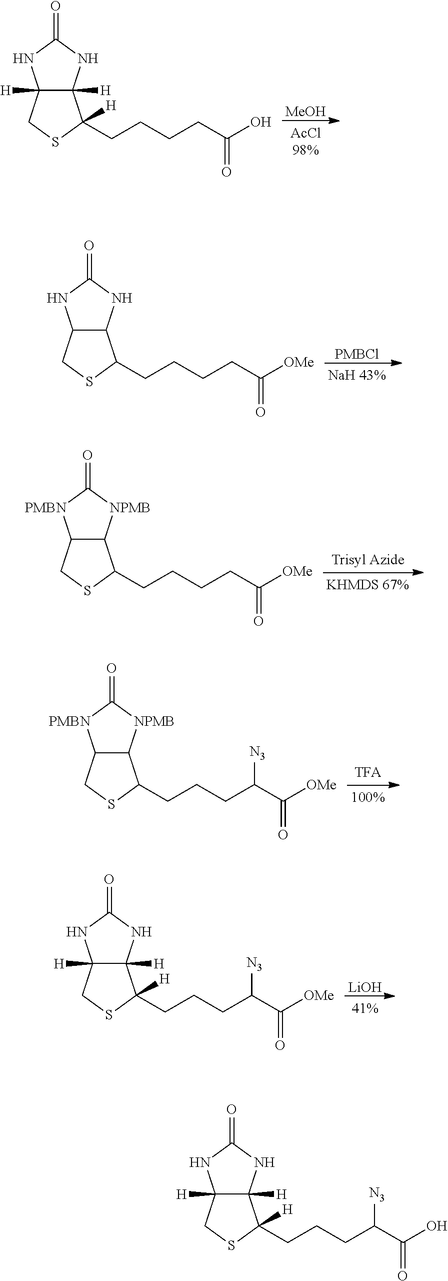

### (i) Synthesis of (+)-Biotin-methyl ester

### (Aubert, D. G. L. University of Nottingham PhD Thesis, 2004):

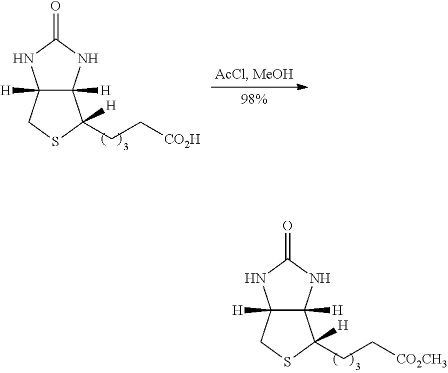

Acetyl chloride (0.61 mL, 8.51 mmol) was added to a solution of (+)-biotin (490 mg, 2.0 mmol) in anhydrous methanol (10 mL) under an inert atmosphere of nitrogen at 0° C. The solution was stirred at room temperature overnight, then the solvent was removed in vacuo to give a pale yellow solid. This was purified by flash chromatography, eluting with dichloromethane:methane (15:1) to give the product as a white powder, 505 mg, 1.96 mmol, yield 98%. M.p. 162.0-162.3° C. [α]²⁵_{D}+49.5 (c 1.0, CHCl₃); ¹H NMR (400 MHz, CDCl₃): δ 5.77 (br s, 1H, NH), 5.39 (br s, 1H, NH), 4.51 (dd, J=7.2, 4.8 Hz, 1H, NCH), 4.31 (dd, J=6.8, 4.8 Hz, 1H, NCH), 3.66 (s, 3H, CH₃), 3.17-3.14 (m, 1H, SCH), 2.91 (dd, J=12.8, 5.1 Hz, 1H, SCH_{A}H), 2.74 (d, J=12.8 Hz, 1H, SCH_{B}H), 2.34 (t, J=7.6 Hz, 2H, CH₂CO), 1.78-1.57 (m, 4H, (CH₂)₂), 1.53-1.34 (m, 2H, CH₂); ¹³C NMR (100 MHz, MeOH-d4): δ 175.82, 165.96, 63.89, 62.22, 56.87, 52.01, 40.83, 34.52, 29.68, 29.41, 25.87; FI—IR (in CHCl₃ solution): 3272.5, 2923.4, 1743.2, 1698.6, 1464.2 cm⁻¹; ESI-MS m/z 259.1054 ([M+H]⁺); HRMS calcd. for C₁₁H₁₉N₂O₃S ([M+H]⁺) 259.1116. Found 259.1110.

### (ii) Synthesis of N,N′-p-methoxybenzylbiotin methyl ester

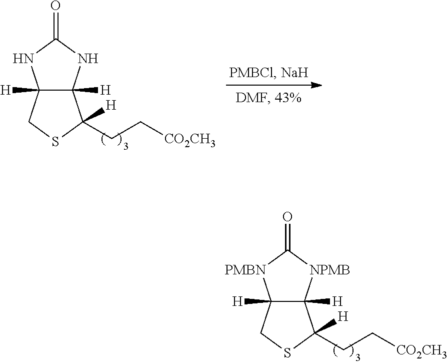

The solution of biotin methyl ester prepared in step (i) (517 mg, 2.0 mmol) in anhydrous DMF (12 mL) was added slowly to the suspension of NaH (60% dispension in mineral oil) (240 mg, 6.0 mmol) in anhydrous DMF (9.6 mL) under an inert atmosphere of nitrogen at 0° C., 20 min later, 4-methoxy benzyl chloride (0.940 g, 6.0 mmol) was added slowly to the reaction mixture. After addition, the mixture was stirred at room temperature for 4 h, then neutralized with saturated ammonium chloride aqueous solution. The solvent was removed by evaporation in vacuo, and the residue was dissolved in ethyl acetate, washed successively with water, saturated sodium chloride solution and the organic phase was dried over anhydrous sodium sulfate. A pale yellow oil was obtained after evaporation in vacuo. This was purified with flash chromatography, eluted with ethyl acetate:petroleum ether (1:2) to give colorless oil, 425 mg, 0.85 mmol, yield 43%. [α]²³_{D}-43.1 (c 1.05, CHCl₃); ¹H NMR (400 MHz, CDCl₃): δ 7.17 (t, J=8.4 Hz, 4H, Ar—H), 6.85 (d, J=8.4 Hz, 4H, Ar′—H), 5.00 (d, J=14.8 Hz, 1H, ArCH_{A}HO), 4.66 (d, J=14.8 Hz, 1H, ArCH_{B}HO), 4.08 (d, J=14.8 Hz, 1H, Ar′CH_{A}HO), 3.88 (d, J=14.8 Hz, 1H, Ar′CH_{B}HO), 3.97-3.81 (m, 2H, NCH and N′CH), 3.80 (s, 6H, CH₃OAr and CH₃OAr′), 3.68 (s, 3H, COOCH₃), 3.11-3.03 (m, 1H, SCH), 2.32 (dd, J=12.4, 4.0 Hz, 1H, SCH_{A}H), 2.30 (dd, J=12.4, 6.0 Hz, 1H, SCH_{B}H), 2.31 (td, J=6.4, 2.0 Hz, 2H, CH₂CO), 1.75-1.23 (m, 6H, (CH₂)₃); ¹³C NMR (100 MHz, CDCl₃): δ 173.89, 160.91, 159.01, 158.98, 129.52, 129.46, 128.88, 128.78, 113.96, 113.91, 62.38, 60.96, 55.19, 54.13, 51.48, 47.23, 45.90, 34.71, 33.79, 28.54, 28.38, 24.57; FI—IR (in CHCl₃ solution) 3606.8, 2936.6, 2838.0, 2400.0, 1729.0, 1683.6, 1612.1, 1586.0, 1456.8, 1381.2 cm⁻¹; ESI-MS m/z 499.2391 ([M+H]⁺); HRMS calcd. for C₂₇H₃₄N₂NaO₅S ([M+Na])⁺) 521.2086. Found 521.2094.

### (iii) Synthesis of N,N′-p-methoxybenzyl-2-azidobiotin methyl ester

(Pearson, A. J., Zhang, P. and Lee. K. J. Org. Chem., 1996. 61, 6581-6586)

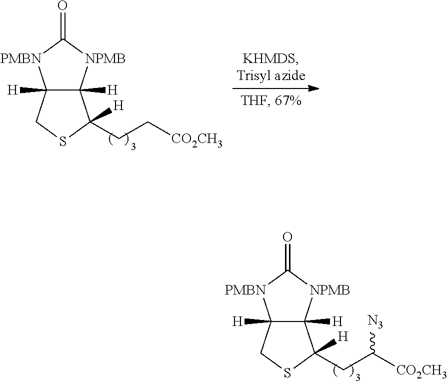

KHMDS (0.4 mL, 0.5 N in toluene) was added slowly to the solution of N,N′-p-methoxybenzyl biotin methyl ester (73 mg, 0.15 mmol) in anhydrous THF (3.0 mL) at −78° C. under the inert atmosphere of argon, 30 min later, the precooled solution of trisyl azide (53 mg, 0.17 mmol) in anhydrous THF (1.6 mL) at −78° C. was added to the reaction solution by canula, 1 h later, glacial acetic acid (0.02 mL) was added, and stirred at room temperature for 3 h, the solution became white slurry. After removal of solvent under vacuum, the residue was dissolved in dichloromethane, then washed successively with saturated sodium bicarbonate, water and saturated sodium chloride solution, then dried over anhydrous sodium sulfate. Pale yellow oil was obtain after evaporation under vacuum, it was purified by flash chromatography, eluted with ethyl acetate:petroleum ether (1:2) to give a colorless oil, 55 mg, 0.10 mmol, yield 67%. NMR (400 MHz, CDCl₃): δ 7.18-7.14 (m, 4H, Ar—H), 6.83 (d, J=8.4 Hz, 4H, Ar′—H), 5.00 (d, J=14.8 Hz, 0.6H, ArCH_{A}H) (major isomer), 4.96 (dd, J=14.8, 2.4 Hz, 0.4H, ArCH_{A}H) (minor isomer), 4.67 (d, J=15.2 Hz, 0.6H, ArCH_{B}H) (major isomer), 4.66 (d, J=14.8 Hz, 0.4H, ArCH_{B}H) (minor isomer), 4.07 (d, J=15.2 Hz, 1H, Ar′CH_{A}H), 3.87 (d, J=15.2 Hz, 1H, Ar′CH_{B}H), 3.97-3.73 (m, 8H), 3.67 (s, 3H, COOCH₃), 3.10-3.00 (m, 1H, SCH), 2.70-2.60 (m, 2H, SCH₂), 2.30 (td, J=7.2, 2.0 Hz, 1H, CH₂CO), 1.90-1.20 (m, 6H, (CH₂)₃); ¹³C NMR (100 MHz, CDCl₃): δ 173.91, 170.81, 160.92, 160.85, 159.07, 159.02, 158.99, 129.55, 129.53, 129.47, 128.88, 128.79, 128.69, 113.99, 113.98, 113.94, 113.92, 62.64, 62.60, 62.39, 61.76, 61.68, 60.97, 60.94, 55.20, 54.14, 53.69, 52.62, 52.60, 51.49, 47.29, 47.24, 45.98, 45.91, 34.72, 34.66, 33.81, 31.00, 28.55, 28.39, 28.08, 25.20, 25.09, 24.58 (it is a mixture of two isomers); FI—IR (in CHCl₃ solution) 2935.7, 2838.2, 2109.0, 1732.3, 1682.8, 1612.4, 1456.4, 1356.4, 1038.2 cm⁻¹; ESI-MS m/z 540.2253 ([M+H]⁺); HRMS calcd. for C₂₇H₃₃N₅NaO₅S ([M+Na]⁺) 562.2095. Found 562.2095.

### (iv) Synthesis of 2-azidobiotin methyl ester

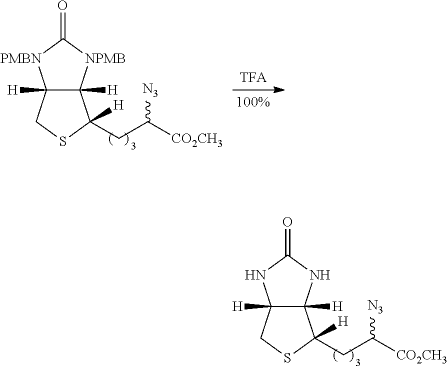

N,N′-p-methoxybenzylbiotin methyl ester (104 mg, 0.19 mmol) was dissolved in trifluoroacetic acid (1.0 mL), and refluxed for 1 h, then trifluoacetic acid was removed by evaporation under vacuum to give a pale red oil. It was purified with flash chromatography, eluted with ethyl acetate:petroleum ether (2:1), colourless sticky oil was obtained, 57 mg, 0.19 mmol, yield 100%. ¹H NMR (400 MHz, MeOH-d4): δ 4.48 (dd, J=7.6, 4.8 Hz, 1H, NCH), 4.29 (dd, J=8.0, 4.8 Hz, 1H, N′CH), 3.65 (s, 3H, OCH₃), 3.22-3.15 (m, 1H, SCH), 2.92 (dd, J=12.8, 4.2 Hz, 1H, SCH_{A}H), 2.70 (d, J=12.8 Hz, 1H, SCH_{B}H), 2.34 (t, J=7.2 Hz, 1H, CHN₃), 1.90-1.40 (m, 6H, (CH₂)₃) (it is inseparable from its epimer); ¹³C NMR (100 MHz, MeOH-d4): δ 175.92, 166.33, 63.40, 61.65, 56.95, 52.01, 41.02, 34.56, 29.70, 29.46, 25.92; FI—IR (in CHCl₃ solution) 3466.4, 2929.5, 2109.6, 1707.5, 1456.1, 1331.5 cm⁻¹; ESI-MS m/z 300.1150 ([M+H]⁺); HRMS calcd. for C₁₁H₁₈N₅O₃S ([M+H]⁺) 300.1125. Found 300.1111.

### (v) Synthesis of 2-azidobiotin

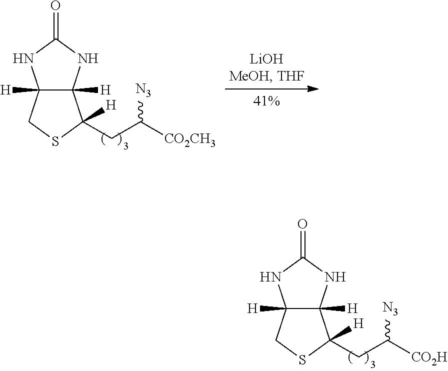

2-azidobiotin methyl ester (52 mg, 0.17 mmol) was dissolved in methanol (0.7 mL) and THF (0.7 mL), then the mixture was cooled to 0° C., lithium hydroxide solution (0.9 M in water) (0.77 mL, 0.7 mmol) was added, then stirred at 4° C. for 4 h. Solvent was removed with evaporation under vacuum, the residue was purified with flash chromatography, eluted with dichloromethane:methanol (10:1), containing 0.25% TFA, to give white solid, 20 mg, 0.47 mmol, yield 41%. NMR (400 MHz, MeOH-d₄): δ 4.48 (dd, J=7.6 Hz, 4.0 Hz, 1H, NCH), 4.30 (dd, J=7.6, 4.4 Hz, 1H, N′CH), 3.96 (dd, J=8.4, 5.2 Hz, 1H, CHN₃), 3.21 (dt, J=9.2, 5.2 Hz, 1H, SCH), 2.93 (dd, J=12.4, 4.8 Hz, 1H, SCH_{A}H), 2.71 (d, J=12.8 Hz, 1H, SCH_{A}H), 1.92-1.80 (m, 1H), 1.80-1.68 (m, 2H), 1.68-1.50 (m, 3H); ¹³C NMR (125 MHz, MeOH-d4): δ 175.10, 166.14, 63.851, 63.374, 61.59, 56.88, 41.06, 32.56 (major isomer), 32.49 (minor isomer)) 29.37 (major isomer), 29.22 (minor isomer), 26.66 (major isomer), 26.53 (minor isomer); FI—IR (KBr solid) 3375.40, 2932.65, 2865.26, 2112.17, 1725.17, 1630.18, 1219.94 cm⁻¹; ESI-MS m/z 284.0766 ([M−H]⁻); HRMS calcd. for C₁₀H₁₄N₅O₃S ([M−H]⁻) 284.0823. Found 284.0826.

### Example 1B

### Optimisation of the Method of Preparation of 2-Azidobiotin Using the Synthetic Route of Example 1A

The synthetic scheme used in (A) above was carried out again to improve yield to 17%, as outlined below:

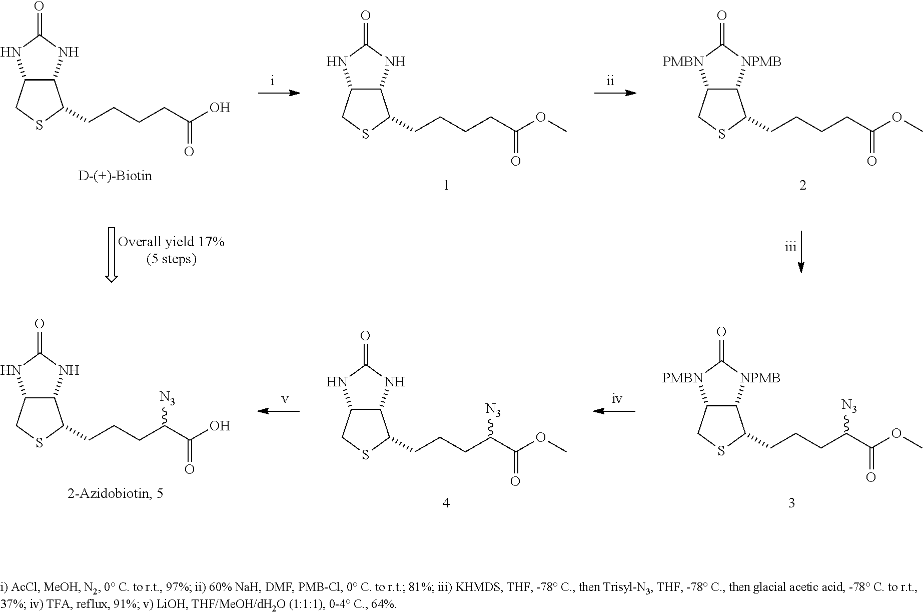

### (i) The Synthesis of Biotin Methyl Ester (1)

D-(+)-Biotin (1.80 g, 7.37 mmol) was suspended in anhydrous methanol (30 ml) under an atmosphere of nitrogen, and cooled to 0° C. prior to the drop wise addition of acetyl chloride (4 equiv., 29.59 mmol, 2.1 ml). The mixture was stirred at ambient temperature overnight prior to removal of the solvent in vacuo to yield a yellow solid which was purified by flash chromatography (1 MeOH:15 DCM; rf˜0.29) to yield 1 as a white crystalline solid (1.84 g, 7.12 mmol, 97%). NMR (400 MHz, CDCl₃) δ 5.69 (1H br s, NH), 4.54 (1H, ddd, J=7.8, 5.0, 1.0, NCH), 4.34 (1H, dd, J=7.8, 4.6, NCH), 3.69 (3H, s, OCH₃), 3.21-3.16 (1H, m, SCH), 2.94 (1H, dd, J=12.8, 5.0, SCH₂), 2.77 (1H, d, J=12.8, SCH₂), 2.36 (2H, t, J=7.5, CH₂CO), 1.80-1.62 (4H, m, 2CH₂), 1.55-1.38 (2H, m, CH₂) ppm. ¹³C NMR (100 MHz, CDCl₃) δ 174.1, 163.4, 62.0, 60.2, 55.3, 51.6, 40.5, 33.7, 28.3, 28.2, 24.8 ppm. Mp. 162-163° C. (Lit^{2b} 162.0-162.3° C.). FT-IR (KBr solid) νₘₐₓ 3274.9 (NH), 2922.2 (CH), 1745.0 (C═O ester), 1708.7 (C═O urea) cm⁻¹. HRMS m/z calc. C₁₁H₁₈N₂O₃SNa [M+Na]⁺ requires 281.0930. Found 281.0931.

### (ii) The synthesis of N,N′-p-methoxybenzylbiotin methyl ester (2)

Biotin methyl ester 1 (1.54 g, 5.96 mmol) in anhydrous DMF (35 ml) was added via cannula to a suspension of NaH (3 equiv., 17.90 mmol, 716 mg) in anhydrous DMF (20 ml) at 0° C. under an atmosphere of nitrogen. The suspension was stirred for 20 mins prior to the drop wise addition of p-methoxybenzyl chloride (3 equiv., 17.90 mmol, 2.43 ml) over 10 mins. The mixture was stirred at 0° C. for 5 min prior to stirring at ambient temperature overnight. Aqueous NH₄Cl (sat.; 20 ml) was added and all solvents removed in vacuo. The residue was dissolved in EtOAc (20 ml) and washed with water (2×20 ml) and brine (20 ml) prior to drying (MgSO₄) to yield a yellow oil. Purification by flash chromatography (1-33% EtOAc in PE, rf˜0.32; 2-33% Et₂O in PE, rf˜0.28) yielded two samples of 2 as a colourless oil, the first (1.50 g, 3.02 mmol, 51%) having approximate NMR purity of 95% and the second (901 mg, 1.81 mmol, 30%) having approximate NMR purity of 86%. ¹H NMR (400 MHz, CDCl₃) δ 7.19 (4H, t, J=8.4, 4ArH), 6.87 (4H, d, J=8.4, 4ArH), 5.00 (1H, d, J=15.2, NCH₂Ar), 4.67 (1H, d, J=15.2, NCH₂Ar), 4.11 (1H, d, J=15.2, NCH₂Ar), 4.00-3.84 (3H, m, NCH₂Ar, 2NCH), 3.81 (6H, s, 2ArOCH₃), 3.70 (3H, s, CO₂CH₃), 3.12-3.05 (1H, m, SCH), 2.74 (1H, dd, J=12.5, 4.2, SCH₂), 2.68 (1H, dd, J=12.5, 6.2, SCH₂), 2.31 (2H, td, J=7.3, 2.0, CH₂CO), 1.73-1.32 (6H, m, 3CH₂) ppm. ¹³C NMR (100 MHz, CDCl₃) δ 173.9, 161.0, 159.2, 159.1, 129.6, 129.6, 129.0, 128.9, 114.1, 114.0, 62.6, 61.1, 55.3, 54.2, 51.5, 47.3, 46.0, 34.8, 33.9, 28.6, 28.5, 24.7 ppm. FT-IR (NaCl liquid) νₘₐₓ 2997.8, 2934.7, 2858.3, 2835.7 (CH), 1734.3 (C═O ester), 1697.1 (C═O urea), 1611.5, 1584.8, 1416.9 (Ar C—C) cm⁻¹. HRMS m/z calc. C₂₇H₃₄N₂O₅SNa [M+Na]⁺ requires 521.2081. Found 521.2079.

### (iii) The synthesis of N,N′-p-methoxybenzyl-2-azidobiotin methyl ester 3

N,N′-p-methoxybenzylbiotin methyl ester 2 (512 mg, 1.03 mmol) and trisyl azide (1.15 equiv., 365 mg, 1.18 mmol) were dried at room temperature under vacuum in oven dried glassware for 1 h prior to purging with argon. 2 was dissolved in anhydrous THF (25 ml) and cooled to −78° C. prior to the drop wise addition of KHMDS (1.33 equiv., 1.37 mmol, 2.73 ml). The mixture was stirred for 30 mins prior to the addition of pre-cooled trisyl azide in THF (1.5 ml, −78° C.) via cannula and stirring was continued for 1 h. Glacial acetic acid (2.4 equiv., 2.46 mmol, 0.14 ml) was added and the mixture allowed to warm to ambient temperature over 4 h. The solvent was removed in vacuo and the product purified by flash chromatography (3% acetone in DCM, rf˜0.35) to yield 3 as a colourless oil (203 mg, 0.38 mmol, 37%) and recovered 2 (118 mg, 23%). ¹H NMR (400 MHz, CDCl₃) δ 7.19 (4H, dd, J=8.6, 5.4, 4ArH), 6.87 (4H, d, J=8.6, 4ArH), 4.95 (1H, dd, J=15.0, 2.8, NCH₂Ar), 4.66 (1H, d, J=15.0, NCH₂Ar), 4.11 (1H, d, J=15.0, NCH₂Ar), 4.00-3.85 (4H, m, NCH₂Ar, 2NCH, CHN₃), 3.85-3.80 (9H, m, 2ArOCH₃, CO₂CH₃), 3.11-3.02 (1H, m, SCH), 2.77-2.66 (2H, m, SCH₂), 1.92-1.24 (6H, m, 3CH₂) ppm. ¹³C NMR (100 MHz, CDCl₃) δ 170.9, 170.9, 160.9, 159.2, 159.1, 129.6, 129.6, 128.9, 128.8, 114.1, 114.1, 62.8, 62.8, 61.9, 61.8, 61.1, 55.3, 53.7, 52.7, 52.6, 47.4, 47.4, 46.1, 34.7, 31.1, 28.2, 25.2, 25.1 ppm. FT-IR (NaCl liquid) νₘₐₓ 3000.0, 2932.2, 2861.0, 2835.8 (CH), 2106.4 (N₃), 1742.0 (C═O ester), 1691.2 (C═O urea), 1611.4, 1584.8, 1512.0 (Ar C—C) cm⁻¹. HRMS m/z calc. C₂₇H₃₃N₅O₅SNa [M+Na]⁺ requires 562.2095. Found 562.2088.

### (iv) The synthesis of 2-azidobiotin methyl ester (4)

N,N′-p-methoxybenzyl-2-azidobiotin methyl ester 3 (203 mg, 0.38 mmol) was dissolved in TFA (2 ml) and heated to reflux for 1 h. The TFA was removed in vacuo and the resulting red residue purified by flash chromatography (5% MeOH in EtOAc, rf˜0.17) to yield 4 as a colourless oil (102 mg, 0.34 mmol, 91%). ¹H NMR (400 MHz, CDCl₃) δ 6.19 (0.5H, s, 0.5NH), 6.06 (0.5H, S, 0.5NH), 5.72 (1H, br s, NH), 4.48-4.40 (1H, m, NCH), 4.28-4.22 (1H, m, NCH), 3.87-3.79 (1H, m, CHN₃), 3.73 (3H, s, CO₂CH₃), 3.12-3.04 (1H, m, SCH), 2.85 (1H, dd, J=12.8, 5.0, SCH₂), 2.66 (1H, d, J=12.8, SCH₂), 1.90-1.34 (6H, m, 6CH₂) ppm. ¹³C NMR (100 MHz, CDCl₃) δ 171.1, 171.0, 164.3, 62.2, 62.1, 61.8, 61.7, 60.3, 55.4, 55.3, 52.7, 40.5, 31.2, 31.1, 28.1, 28.0, 25.3, 25.2 ppm. FT-IR (KBr solid) νₘₐₓ 3234.5 (NH), 2944.9, 2863.7 (CH), 2116.6 (N₃), 1698.7 (C═O urea) cm⁻¹. HRMS m/z calc. C₁₁H₁₇N₅O₃SNa [M+Na]⁺ requires 322.0944. Found 322.0941.

### (v) The synthesis of 2-azidobiotin (5)

2-Azidobiotin methyl ester 4 (81 mg, 0.27 mmol) was dissolved in a mixture of MeOH and THF (3.6 ml, 1:1) and cooled to 0° C. prior to the drop wise addition of LiOH (6 equiv., 1.61 mmol, added as 1.8 ml of a 0.9 M aqueous solution) and the mixture was stirred for 4 h at 4° C. The solvents were removed in vacuo and the residue dissolved in NaHCO₃ (sat., 5 ml, diluted 1:1 in water) and any impurities extracted into DCM (3×5 ml). The aqueous was acidified (1M HCl, pH 2) and 2-azidobiotin allowed to crystallise at 4° C. overnight to yield 5 as white crystalline needles (41 mg, 0.14 mmol, 53%). An additional crop of 5 was obtained as a cream powder (8 mg, 0.03 mmol, 11%) by evaporating the aqueous and crystallising the residue from 1M HCl (aq.) at 4° C. overnight. ¹H NMR (400 MHz, d₆-DMSO) δ 13.29 (1H, br s, CO₂H), 6.43 (1H, s, NH), 6.35 (1H, s, NH), 4.35-4.27 (1H, m, NCH), 4.17-4.11 (1H, m, NCH), 4.11-4.05 (1H, m, CHN₃), 3.17-3.08 (1H, m, SCH), 2.84 (1H, dd, J=12.4, 5.2, SCH₂), 2.59 (1H, d, J=12.4, SCH₂), 1.85-1.33 (6H, m, 3CH₂) ppm. ¹³C NMR (100 MHz, d₆-DMSO) δ 172.3 (minor), 172.3 (major), 163.1, 61.7, 61.5, 61.4, 59.7 (major), 59.6 (minor), 55.7 (minor), 55.6 (major), 31.2 (major), 31.1 (minor), 28.3 (major), 28.2 (minor), 25.5 (major), 25.4 (minor) ppm. Mp. 209-210° C. FT-IR (KBr solid) νₘₐₓ 3298.7 (NH), 2927.7 (CH), 2497.4 (OH), 2112.7 (N₃), 1725.9 (C═O acid), 1626.4 (C═O urea) cm⁻¹. HRMS m/z calc. C₁₀H₁₄N₅O₃S [M−H]⁻ requires 284.0823. Found 284.0809.

The methyl ester formation to yield 1 was achieved by treating a suspension of biotin in anhydrous methanol with acetyl chloride in excellent yield after purification. Subsequent N,N′-ureido protection with PMB-chloride to yield 2 proved more troublesome, due mainly to the purity of the PMB-Cl starting material or its subsequent decomposition under the reaction conditions. NMR analysis of the PMB-Cl demonstrated it to be of good purity, however the same was not observed by TLC (4 spots). Initial purification of the reaction product by flash chromatography (33% PE in EtOAc; rf˜0.31) proved unsuccessful. A second column using the mobile phase (33% EtOAc in PE; rf˜0.32) yielded 2 which was TLC pure but NMR analysis demonstrated the presence of a mixture of 2 and an aromatic impurity (i.e. two co-eluting TLC spots), assumed to be a related breakdown product of PMB-Cl. A third column (33% PE in Et₂O; rf˜0.28) was used to purify the PMB protected product 2, however as the impurity was not clearly observed by TLC, ¹H NMR was used to establish the purity of the product containing fractions prior to concentration. This yielded several portions of 2 with estimated ¹H NMR purities of 95% (50% non-adjusted yield) and 86% (30% non-adjusted yield), with other less pure product containing fractions discarded.

Completion of the azidation step had previously proved difficult to successfully achieve, other than when conducted by Y. Q. Yang (not yet published). THF was freshly distilled from sodium/benzophenone and stored under an inert atmosphere over 4 Å molecular sieves, and new batches of KHMDS and Trisyl azide were purchased. All glassware/cannulas used in these experiments were oven dried prior to use and cooled to ambient temperature in a desicator/under a stream of argon respectively. In addition, anhydrous conditions were maintained by completing the reaction in an atmosphere of argon, and the N,N′-p-methoxybenzylbiotin methyl ester (2) and trisyl azide were dried under vacuum at ambient temperature for 1 hour prior to purging with argon before using in the experiment.

Several small scale reactions (˜30 mg) were conducted to learn more about the reaction conditions and their effects upon the success of the reaction, as detailed in Table 1 below:

TABLE 1

| | | | | Yield by MS |
|---|---|---|---|---|
| Rxn | Enolization | Azidation | Acid Quench | SM (2):Product (3) |
| A | 1.33x | 1.15x Tri-N₃, | 2.4x H⁺, −78° C. to r.t., 3 h | 25:121 |
| | KHMDS, −78°, | −78 to −60° C., | r.t., O/N | (83% conversion) |
| | 30 min | 1 h | | Polymer product |
| B | 1.33x KHMDS, | 1.15x Tri-N₃, | 2.4x H⁺, −78° C. to r.t., 4 h | 63:137 |
| | −78°, 30 min | −78 to −60° C., | | (69% conversion) |
| | | 1 h | | |
| C | 1.33x | 1.15x Tri-N₃, | 2.4x H⁺, −78° C. to r.t., 3 h | 126:35 |
| | KHMDS, −78°, | −78 to −50° C., | r.t., O/N | (22% conversion) |
| | 30 min | 1 h | | 129:38 |
| | | | | (23% conversion) |
| D | 1.33x | 1.15x Tri-N₃, | 2.4x H⁺, −78° C. to r.t., 3 h | 42:136 |
| | KHMDS, −78°, | −78° C., 1 h | r.t., O/N | (76% conversion) |
| | 30 min | | | Polymer |
| E | 1.33x | 1.15x Tri-N₃, | 2.4x H⁺, −78° C. to r.t., 3 h | 47:100 |
| | KHMDS, −78°, | −78° C., 2 min | r.t., O/N | (68% conversion) |
| | 30 min | | | Polymer |

It was observed from this that in all cases full conversion of 2 to 3 was not obtained, however in no case was the diazo-by-product identified by MS (Evans, D. A. et al., JACS, 1990, 112, 4011-4030). To attempt to optimise the conditions of the reaction to maximise the conversion of 2 to 3, both the temperature and time period of the azidation step were investigated. It was observed that the reaction demonstrated some temperature tolerance up to −60° C. (A and B), however warming the azidation step to −50° C. proved detrimental to azide transfer (C). The reaction time period demonstrated that 1 hour azidation was optimal (D) over the previously reported 2 mins (E) (Evans supra) and that leaving the reaction for longer than 3-4 hours at ambient temperature during the glacial acetic acid mediated intermediate breakdown (A, C, D and E) often resulted in an unidentified polymer product by MS (single polymer unit mass 74). Hence the optimal and most reproducible reaction conditions were thought to be those of reaction D involving treatment with KHMDS (1.33×) at −78° C. for 30 mins, followed by trisyl azide (1.15×) at −78° C. for 1 hour prior to glacial acetic acid addition (2.4×) and warming to ambient temperature over 3-4 hours (Yang et al; not yet published and Pearson, A. J. et al., JOC, 1996, 61, 6581-6586). When these conditions were employed in the reaction, a mixture of the N,N′-p-methoxybenzylbiotin methyl ester 2 and N,N′-p-methoxybenzyl-2-azidobiotin methyl ester 3 was obtained which proved difficult to separate by flash chromatography. Attempts utilising the mobile phase (33% EtOAc in PE) did not result in separation. After much effort, the optimal solvent for the separation of 2 and 3 was found (3% acetone in DCM; rf 2˜0.39, rf 3˜0.30), and upon scale up this reaction yielded 3 in 37% yield with 23% recovered 2.

PMB deprotection was achieved in refluxing TFA to yield 4 in 91%. However the product proved too polar for the chromatography mobile phase (33% PE in EtOAc) and an alternative was employed (5% MeOH in EtOAc; rf˜0.17). Finally, saponification of the methyl ester of 4 was achieved using LiOH and upon acidification of the aqueous reaction liquor the 2-azidobiotin product 5 spontaneously crystallised from solution.

### Example 2

### Alternative Method for Preparation of 2-Azidobiotin Using 2-Oxazolidinone

Many synthetic routes were attempted to directly add the non-chiral Evans auxiliary analogue 2-oxazolidinone to biotin. These included routes which use the acid chloride of biotin prior to attempted displacement with the auxilary, peptide coupling reactions (DCC, EDCI, HBTU), mixed anhydride methods (AcCl, Piv-Cl) and displacement of the activated N-hydroxysuccinimide product. However all of these methods proved unsuccessful mainly due to the inherent insolubility of the biotin starting material and the resulting reaction products in many organic solvents.

Subsequently, the synthetic route 2 illustrated below was proposed since the PMB-protected N,N′-ureido functionality offered significant benefits to compound solubility. Hence, route 2 initially followed that previously described for route 1 to yield the PMB-protected biotin methyl ester 2. At this stage, the less pure fraction of 2 (approx. 86% by ¹H NMR) was subjected to saponification to yield N,N′-p-methoxybenzylbiotin 6 in 91-100% yield. The exact yield of this step is unknown since the extent of the impurity present on the starting material is not fully known, however during the isolation of the product all impurities were removed by acid/base extraction. This affords a quick, simple purification method for material which proved difficult to purify by traditional flash chromatography methods in the previous step.

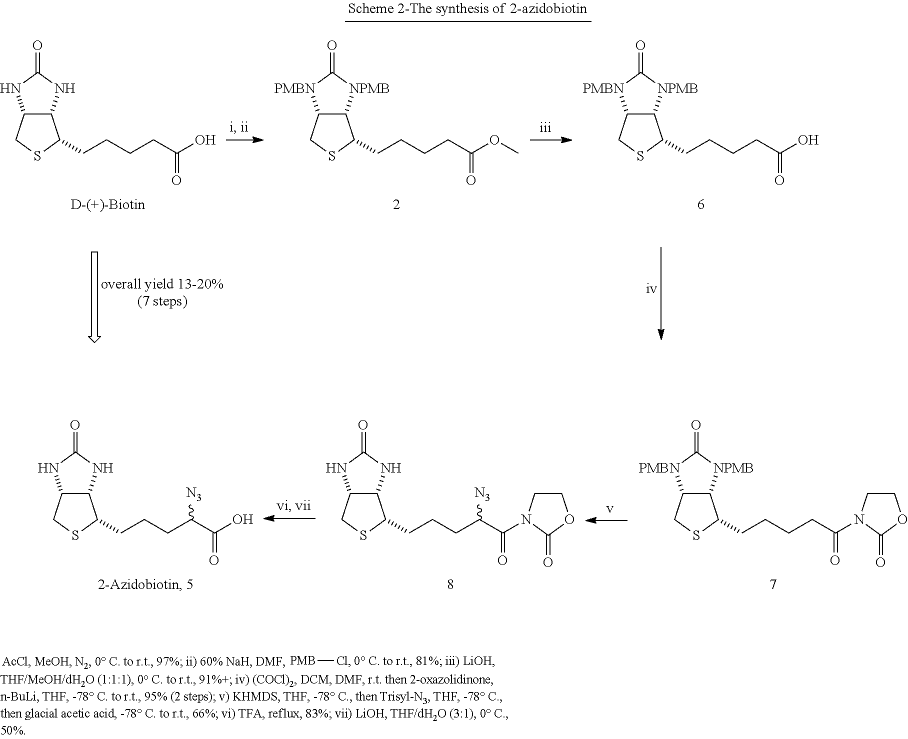

Steps (i) and (ii) correspond to steps (i) and (ii) in Example 1B.

### (iii) The synthesis of N,N′-p-methoxybenzylbiotin (6)

N,N′-p-methoxybenzylbiotin methyl ester 2 (approx NMR purity 86%, 788 mg, 1.58 mmol) was dissolved in a mixture of MeOH and THF (20 ml, 1:1) and cooled to 0° C. prior to the drop wise addition of LiOH (6 equiv., 9.62 mmol, added as 9.6 ml of a 1 M aqueous solution) and the mixture was stirred for 4 h at 0° C. and overnight at ambient temperature. The solvents were removed in vacuo and the residue dissolved in NaHCO₃ (sat., 50 ml, diluted 1:1 in water) and any impurities extracted into EtOAc (3×10 ml). The aqueous was acidified (conc. HCl, pH 2) and the product extracted into EtOAc (5×40 ml). The organic portion was washed with brine (20 ml) and dried (MgSO₄) to yield 6 as a colourless oil (697 mg, 1.44 mmol, 91%+). Rf˜0.00 (1 MeOH:15 DCM). ¹H NMR (400 MHz, CDCl₃) δ 7.19 (4H, dd, J=8.7, 6.8, 4ArH), 6.87 (4H, d, J=8.7, 4ArH), 4.99 (1H, d, J=15.1, NCH₂Ar), 4.67 (1H, d, J=15.1, NCH₂Ar), 4.11 (1H, d, J=15.1, NCH₂Ar), 4.00-3.83 (3H, m, NCH₂Ar, 2NCH), 3.82 (6H, s, 2ArOCH₃), 3.12-3.04 (1H, m, SCH), 2.78-2.65 (2H, m, SCH₂), 2.37 (2H, td, J=7.1, 2.8, CH₂CO), 1.77-1.30 (6H, m, 3CH₂) ppm. ¹³C NMR (100 MHz, CDCl₃) δ 178.3, 161.0, 159.2, 159.1, 129.6, 129.6, 128.9, 128.8, 114.1, 114.1, 62.7, 61.1, 55.3, 54.1, 47.4, 46.1, 34.7, 33.8, 28.5, 28.4, 24.4 ppm. FT-IR (NaCl liquid) ν_{Max} 2933.0 (OH and CH), 1691.1 (C═O acid and urea), 1611.4, 1585.0, 1512.2 (Aromatic C—C) cm⁻¹. HRMS m/z calc. C₂₆H₃₁N₂O₅S [M−H]⁻ requires 483.1959. Found 483.1974.

### (iv) The synthesis of 3-(N,N′-p-methoxybenzylbiotinoyl)oxazolidin-2-one (7)

N,N′-p-methoxybenzylbiotin 6 (461 mg, 0.95 mmol) was dissolved in anhydrous DCM (5 ml) under an atmosphere of nitrogen prior to the addition of oxalyl chloride (1.4 equiv., 1.33 mmol, 0.67 ml) and anhydrous DMF (1 drop) and the mixture was stirred at ambient temperature for 1 h prior to the evaporation of the solvent in vacuo. 2-Oxazolidinone (1.1 equiv., 1.05 mmol, 91 mg) was dissolved in anhydrous THF (5 ml) and cooled to −78° C. prior to the drop wise addition of n-BuLi (1.01 equiv of auxiliary, 1.06 mmol, 0.66 ml) over 10 mins. The mixture was stirred at −78° C. for 5 mins prior to the addition of the acid chloride in anhydrous THF (5 ml) via cannula. Stirring was continued at −78° C. for 30 mins and then at ambient temperature for 2 h prior to the removal of the solvent in vacuo to yield a white foam. This was dissolved in EtOAc (50 ml) and washed with NaHCO₃ (3×25 ml), brine (25 ml) and dried (MgSO₄) to yield 7 as a pale yellow foam (496 mg, 0.90 mmol, 95%). Rf˜0.15 (1% MeOH in DCM). ¹H NMR (400 MHz, CDCl₃) δ 7.19 (4H, dd, J=8.7, 5.6, 4ArH), 6.87 (4H, d, J=8.7, 1.2, 4ArH), 5.01 (1H, d, J=15.2, NCH₂Ar), 4.68 (1H, d, J=15.2, NCH₂Ar), 4.46-4.41 (2H, m, CH₂-auxilary), 4.10 (1H, d, J=15.2, NCH₂Ar), 4.06-4.02 (2H, m, CH₂-auxilary), 3.97-3.83 (3H, m, NCH₂Ar, 2NCH), 3.82 (6H, d, J=1.2, 2ArOCH₃), 3.13-3.06 (1H, m, SCH), 2.95 (2H, t, J=7.2, CH₂CO), 2.75 (1H, dd, J=12.6, 4.2, SCH₂), 2.67 (1H, dd, J=12.6, 6.2, SCH₂), 1.78-1.32 (6H, m, 3CH₂) ppm. ¹³C NMR (100 MHz, CDCl₃) δ 173.2, 161.0, 159.1, 159.1, 153.5, 129.6, 129.6, 129.0, 128.9, 114.1, 114.0, 62.5, 62.0, 61.1, 55.3, 55.3, 54.2, 47.3, 46.0, 42.5, 34.9, 34.8, 28.5, 28.5, 24.0 ppm. FT-IR (KBr solid) ν_{Max} 2931.2, 2836.1 (CH), 1778.0 (C═O imide), 1691.3 (C═O urea), 1611.2, 1584.6, 1511.9 (aromatic C—C) cm⁻¹.HRMS m/z calc. C₂₉H₃₆N₃O₆S [M+H]⁺ requires 554.2319. Found 554.2312.

### (v) Synthesis of 3-(N,N′-p-methoxybenzyl-2-azidobiotinoyl) oxazolidin-2-one (8)

3-(N,N′-p-methoxybenzylbiotinoyl)oxazolidin-2-one 7 (91 mg, 0.17 mmol) and trisyl azide (1.15 equiv., 59 mg, 0.19 mmol) were dried at room temperature under vacuum in oven dried glassware for 1 h prior to purging with argon. 7 was dissolved in anhydrous THF (3 ml) and cooled to −78° C. prior to the drop wise addition of KHMDS (1.33 equiv., 0.22 mmol, 0.44 ml). The mixture was stirred for 30 mins prior to the addition of pre-cooled trisyl azide in THF (1.5 ml; −78° C.) via cannula and stirring was continued for 1 h. Glacial acetic acid (2.4 equiv., 0.40 mmol, 23 μl) was added and the mixture allowed to warm to ambient temperature over 4 h. The solvent was removed in vacuo and the product purified by flash chromatography (33% PE in EtOAc, rf˜0.39) to yield 8 as a colourless oil which solidified on standing (65 mg, 0.11 mmol, 66%; residue crystallisable from EtOAc/PE to yield a white solid 37%). ¹H NMR (400 MHz, CDCl₃) δ 7.20 (4H, dd, J=8.6, 1.3, 4ArH), 6.87 (4H, dd, J=8.6, 1.8, 4ArH), 5.03-4.94 (2H, m, NCH₂Ar, CHN₃), 4.68 (1H, d, J=15.1, NCH₂Ar), 4.52 (2H, t, J=8.2, CH₂-auxilary), 4.18-4.03 (3H, m, NCH₂Ar, CH₂-auxilary), 3.98-3.85 (3H, m, NCH₂Ar, 2NCH), 3.82 (6H, d, J=1.8, 2ArOCH₃), 3.17-3.10 (1H, m, SCH), 2.75 (1H, dd, J=12.5, 4.2, SCH₂), 2.69 (1H, dd, J=12.5, 6.2, SCH₂), 1.95-1.46 (6H, m, 3CH₂) ppm. ¹³C NMR (100 MHz, CDCl₃) δ 171.0, 159.1, 153.0, 129.7, 129.6, 129.1, 128.8, 114.1, 114.1, 62.6, 62.6, 60.6, 61.0, 59.9, 55.3, 53.6, 47.3, 46.1, 42.6, 34.8, 30.7, 27.7, 25.1 ppm. Mp. 133-134° C. FT-IR (KBr solid) νₘₐₓ 2930.2, 2834.8 (CH), 2110.4 (N₃), 1783.7 (C═O imide), 1702.33 (C═O imide), 1678.5 (C═O urea), 1609.4, 1512.2 (Ar C—C) cm⁻¹.HRMS m/z calc. C₂₉H₃₄N₆O₆SNa [M+Na]⁺ requires 617.2153. Found 617.2166.

### (vi) The synthesis of 3-(2-azidobiotinoyl)oxazolidin-2-one (9)

3-(N,N′-p-methoxybenzyl-2-azidobiotinoyl)oxazolidin-2-one 8 (137 mg, 0.23 mmol) was dissolved in TFA (1.2 ml) and heated to reflux for 1 h. The TFA was removed in vacuo and the resulting red residue purified by flash chromatography (5% MeOH in DCM, rf˜0.30) to yield 9 as an colourless foam (68 mg, 0.19 mmol, 83%). ¹H NMR (400 MHz, CDCl₃) δ 5.96 (0.5H, s, 0.5NH), 5.73 (0.5H, s, 0.5NH), 5.09 (1H, br s, NH), 5.06 (0.5H, dd, J=4.6, 7.5, CHN₃-isomer), 4.98 (0.5H, dd, J=4.6, 8.8, CHN₃-isomer), 4.58-4.46 (3H, m, NCH, CH₂-auxilary), 4.38-4.32 (1H, m, NCH), 4.15-4.07 (2H, m, CH₂-auxilary), 3.23-3.15 (1H, m, SCH), 2.99-2.91 (1H, m, SCH₂), 2.75 (1H, dd, J=12.7, 4.4, SCH₂), 2.03-1.46 (6H, m, 3CH₂) ppm. FT-IR (KBr solid) ν_{Max} 3405.4 (NH), 2924.7 (CH), 2109.9 (N₃), 1778.4 (C═O imide), 1699.1 (C═O urea) cm⁻¹. HRMS m/z calc. C₁₃H₁₈N₆O₄SNa [M+Na]⁺ requires 377.1002. Found 377.1020.

### (vii) The synthesis of 2-azidobiotin (5)

3-(2-azidobiotinoyl)oxazolidin-2-one 9 (51 mg, 0.14 mmol) was dissolved in THF (3 ml) and cooled to 0° C. prior to the drop wise addition of LiOH (2 equiv., 0.29 mmol, added as 1 ml of a 0.29 M aqueous solution) and stirring continued at 0° C. for 1 h. The organics were removed in vacuo and to the aqueous was added NaHCO₃ (sat., 2 ml) prior to extraction of organic impurities with DCM (3×5 ml). The aqueous was acidified (1M HCl, pH 2) and extracted rapidly with DCM (3×5 ml) prior to allowing 2-azidobiotin to crystallise spontaneously from the aqueous liquor overnight at 4° C. to yield 5 as white crystalline needles (14 mg, 0.05 mmol, 36%). An additional crop of 5 was obtained as a cream powder (5 mg, 0.02 mmol, 14%) by evaporating the aqueous and crystallising the residue from 1M HCl (aq.) at 4° C. overnight. Rf˜0.00 (5% MeOH in DCM). ¹H NMR (400 MHz, d₆-DMSO) δ 6.43 (1H, s, NH), 6.35 (1H, s, NH), 4.34-4.28 (1H, m, NCH), 4.16-4.11 (1H, m, NCH), 4.08-4.03 (1H, m, CHN₃), 3.15-3.09 (1H, m, SCH), 2.84 (1H, dd, J=12.4, 5.2, SCH₂), 2.58 (1H, d, J=12.4, SCH₂), 1.84-1.35 (6H, m, 3CH₂) ppm. ¹³C NMR (100 MHz, d₆-DMSO) δ172.3, 163.2, 61.8, 61.6 (minor), 61.5 (major), 61.4, 59.7 (major), 59.6 (minor), 55.7 (minor), 55.7 (major), 31.3 (major), 31.1 (minor), 28.3 (major), 28.2 (minor), 25.5 (major), 25.4 (minor) ppm. Mp. 209-210° C. FT-IR (KBr solid) νₘₐₓ 3284.1 (NH), 2927.9 (CH), 2495.6 (OH), 2110.5 (N₃), 1723.0 (C═O acid), 1649.1 (C═O urea) cm⁻¹.

HRMS m/z calc. C₁₀H₁₄N₅O₃S [M−H]⁻ requires 284.0823. Found 284.0821.

In this Example, the PMB-protected biotin analogue 6 proved to have significantly enhanced solubility relative to that observed for biotin, allowing standard methods for the addition of the non-chiral auxiliary to be employed. Therefore, the acid chloride of 6 was able to be formed by reaction with oxalyl chloride, followed by in situ displacement with n-BuLi treated 2-oxazolidinone to yield 7 in 95% (2 steps). The ‘optimised’ azidation procedure discussed above was used to incorporate the required azide functionality, and in this case complete consumption of 7 was observed by MS and TLC resulting in simple flash purification (33% PE in EtOAc; rf˜0.39) to yield 8 in 66%. It should be noted that on occasion where complete SM consumption is not observed, the separation of 7 and 8 can still be achieved (33% PE in EtOAc; rf 7˜0.35, rf 8˜0.45). The observed total consumption of 7 demonstrates how the oxazolidinone imide must have enhanced reactivity, presumably through enhanced acidity alpha to the masked carboxylic acid functionality relative to the ester analogue, resulting in simpler purification of the product and enhanced reaction yield. This has been previously demonstrated where specific azidation has occurred alpha to a chiral auxiliary in the presence of an ester functionality (Evans, D. A. et al., JACS, 1990, 112, 4011-4030). Crystallisation of the reaction product was also demonstrated which may allow enantiomeric enrichment when working with asymmetric analogues.

Following completion of the azidation, deprotection to 5 proceeded as described above with no need to employ the oxidative deprotection of the oxazolidinone, favouring standard LiOH mediated saponification instead. It should however be noted that upon PMB deprotection, purification by flash chromatography (5% MeOH in DCM, rf˜0.30) is required as direct saponification of the crude reaction products yielded no 2-azidobiotin 5 following extraction of organic impurities.

The enhanced yield of the azidation reaction for the oxazolidinone product 7 relative to the methyl ester analogue 2, and the high yielding saponification and imide forming reactions utilised in route 2, resulted in a comparable overall yield of 2-azidobiotin 5 of 13-20% by this route even though 7 steps were employed (depending upon the yield of the saponification of 2 to 6). The 2-azidobiotin (5) isolated by this route had excellent analytical purity which was comparable to that observed by the route employed in Example 1.

However, the route of Example 2 does offer several other benefits over that used in Example 1 other than the high yielding azidation step, with the main benefit being the ability to avoid the difficult flash chromatography required for the purification of N,N′-p-methoxybenzylbiotin methyl ester 2, where in this case, a simple extractive purification could be used following methyl ester hydrolysis to 6. The synthesis of 2-azidobiotin by the current route 2 has therefore established the methodology required for the incorporation of chiral auxiliaries to elicit the asymmetric synthesis of R— and S-2-azidobiotin amongst other potential alkylation analogues variable at this position, which may include the 2-propargylbiotin analogues.

### Example 3

### Alternative Method for Preparation of 2-Azidobiotin

An alternative route for the preparation of 2-azidobiotins uses benzyl protecting groups with literature deprotection conditions, as illustrated in the scheme given below:

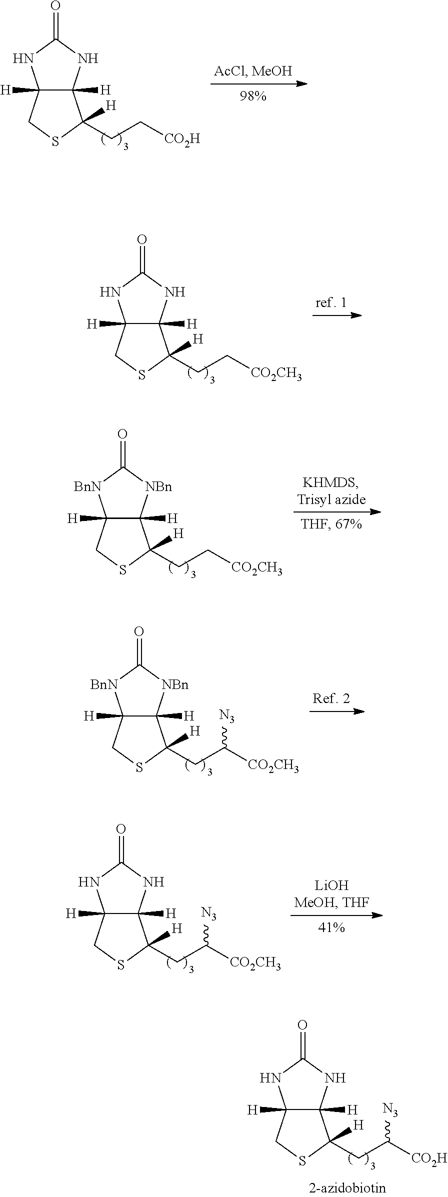

This synthetic route uses the less expensive benzyl bromide to protect the ureido nitrogens, such that the following conditions can be employed:

Ref 1: NaH, DMF, 60 min, 90 deg C.; 2.2 eq BnBr, 24 h. 90 deg C.; H₂O, as described by Kyungsoo Tetrahedron Letters (2007), 48(21), 3685-3688.

Ref 2: 47% HBr, H₂O for 5 hours at 125° C. (or the use of H₂SO₄ and AcOH or MeSO₃H as acids).

Other conditions may be used as is described in the art for the protection and deprotection of the ureido nitrogens.

### Example 4

### Addition of 2-Azidobiotin to an Acceptor Peptide Using BirA Biotin Ligase

The 2-azidobiotin prepared in Example 1 was added to the synthetic acceptor peptide (AP): KKKGPGGLNDIFEAQKIEWHE (SEQ. ID No. 23) using the following incubation conditions:

Ligation condition:50 mM Bicine pH 8.3, 5 mM magnesium acetate, 4 mM ATP, 100 mM AP, 2.9 mM biotin ligase (BirA), 1 mM probe, 30oC, shaker, 1 h. These are those also used for biotin with this enzyme and are modified from those previously reported by Chen et al. (Nature Methods, 2005, 2, 99-104)

Biotin ligase (BirA) is an 321 amino acid, 33.5 kD enzyme derived from E. coli that catalyzes the context-specific conjugation of biotin to a lysine .epsilon.-amine in biotin retention and biosynthesis pathways, as shown in FIG. 3 of the accompanying drawings. This reaction is ATP-dependent. As used herein, wild type biotin ligase refers to a naturally occurring bacterial biotin ligase having wild type biotinylation activity. SEQ ID NO: 1 shown in FIG. 1 of the accompanying drawings represents the amino acid sequence of wild type biotin ligase (GenBank Accession No. M10123) and SEQ ID NO: 2 (shown in FIG. 2) represents the nucleotide sequence of wild type biotin ligase (GenBank Accession No. M10123).

Biotin analogue incorporation can be determined using a variety of assays including but not limited to (1) inhibition of .sup.3H-biotin incorporation, (2) western blot detection of unnatural probe conjugation to cyan fluorescent protein (CFP) bearing a C-terminal Avi-Tag, (3) MALDI mass-spectrometric detection of probe attachment to an Avi-Tag peptide substrate, and (4) HPLC. In the first of these assays, biotin analogue candidates and biotin are incubated together with the biotin ligase its mutants or homologues and the acceptor peptide. Decreases in incorporation of radioactivity are indicative of a biotin analogue that competes effectively with biotin for the biotin ligase or its mutants or homologues activity. In the second of these assays, biotin analogue conjugation to an acceptor peptide is indicated by the use of antibodies specific for the biotin analogue or a label conjugated thereto (e.g., an anti-FLAG antibody or an anti-fluorophore antibody). In the third assay, differences in the molecular weight of the acceptor peptide are indicative of incorporation of the biotin analogue. In the last of these assay, acceptor peptides with longer retention times are indicative of biotin analogue incorporation.

The 2-azidobiotin-acceptor peptide adduct was analysed by HPLC and compared with the HPLC trace of biotin attached to the same peptide using biotin ligase (BirA). The level of conversion of the 2-azidobiotin was demonstrated to be very similar to that of biotin indicating they had similar kinetic parameters, as illustrated in FIGS. 4 and 5 respectively of the accompanying drawings.

### Example 5

### Binding Affinity of 2-Azidobiotin for Avidin

The binding affinities of biotin and 2-azidobiotin (prepared by the method of Example 1) for avidin were examined using isothermal titration calorimetry (ITC) on a Microcal VP-ITC instrument using the following titration conditions: Ligand (biotin or 2-azidobiotin 0.35 mM, Avidin 0.0078 mM Buffer pH 7.4, 20 mM phosphate, 150 mM NaCl). Isothermal Titration Calorimetry Data for 2-azidobiotin with avidin is shown in FIG. 6 of the accompanying drawings.

From the data collected K_{d}˜10⁻⁷ M for 2-azidobiotin with avidin. Whilst considerably lower than that of biotin with avidin (K_{d}=10⁻¹⁴ M)(Green, N. M. (1975) Adv. Protein Chem. 29, 85-133) it is sufficiently strong to allow 2-azidobiotinylated peptides to be separated from non-2-azidobiotinylated peptides and proteins. Unlike the interaction between biotin and avidin which can be considered as being irreversible without denaturing avidin, the 2-azidobiotinylated proteins can then be released from avidin by addition of biotin, other biotin analogues such as the strep tag, HABA or changes in pH or salt concentration in the buffer solution.

### Example 6

### Reaction of 2-Azidobiotin with a Bioorthogonally Functionalised Tag-a Propargyl Functionalised Flourescent Coumarin Derivative

This example demonstrated that the azide group of 2-azidobiotin can be selectively reacted with a propargyl functionalised fluorescent coumarin derivative using a copper catalysed Huisgen cycloaddition reaction (see FIG. 7). This was achieved both in the presence and absence of avidin as shown below.

1) Without protein present:

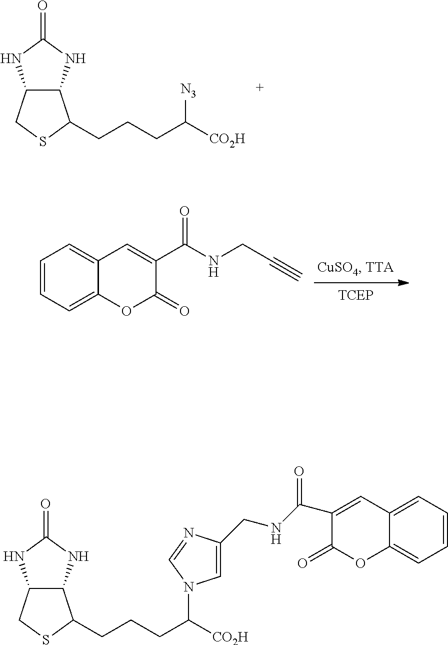

2) With protein (avidin) present:
0.29 mL 2-azidiobiotin solution (3.5 mM in Buffer A) was diluted in 0.71 mL Buffer A, then 1 mL avidin solution (0.21 mM) was added. The mixed solution was put in a shaker (25° C., 100 rpm) for 1 h. Then 0.5 mL alkynyl tag solution (10 mM in dioxane), 0.5 mL CuSO4-TTA solution (10 mM in ^{t}BuOH:H₂O (4:1) solvents), 0.2 mL TCEP.HCl solution (50 mM in H₂O), 0.5 mL NaHCO₃ solution (200 mM in H₂O), 0.25 mL ^{t}BuOH, 1 mL H₂O were added to the reaction mixture. The mixture was put on the shaker (25° C., 100 rpm) for 2 h, then stored at 4° C.
Buffer A: pH 7.4 20 mM phosphate, 150 mM NaCl buffer.

Native gel observed under UV light (FIG. 8b) and stained with coomasie blue (FIG. 8a) indicating that 2-azidobiotin is able to binded to avidin once its azide group has undergone ‘click’ chemistry.

It is to be appreciated that the biotin analogue may be reacted with any appropriate detectable moieties. FIG. 9 of the accompanying drawings illustrates Staudinger-Bertozzi reaction between 2-azidobiotin and a fluorogenic dye that is activated by the Staudinger ligation (G. A. Lemieux, C. L. de Graffenried and C. R. Bertozzi, J. Am. Chem. Soc. 2003, 125, 4708-4709). However, the detectable moiety need not be fluorophore-bearing.

### Example 7

### Comparison of Binding Affinities of Biotin Analogue According to a First Aspect of the Invention with 8-Azidodesthiobiotin, 6-Azidodesthiobiotin and Other Prior Art Analogues

8-Azidodesthiobiotin was prepared using the following series of steps:

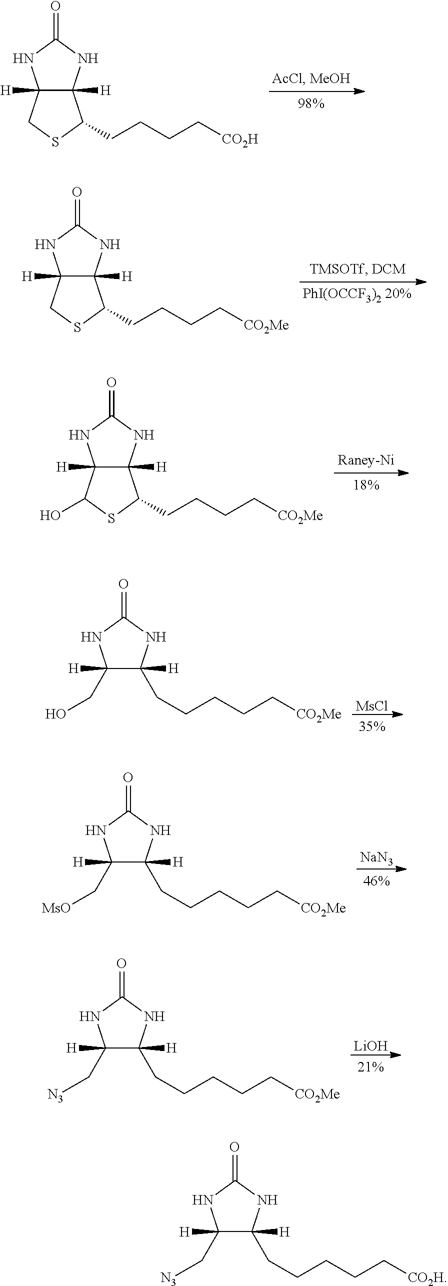

6-Azidodesthiobiotin was prepared using the following sequence of steps:

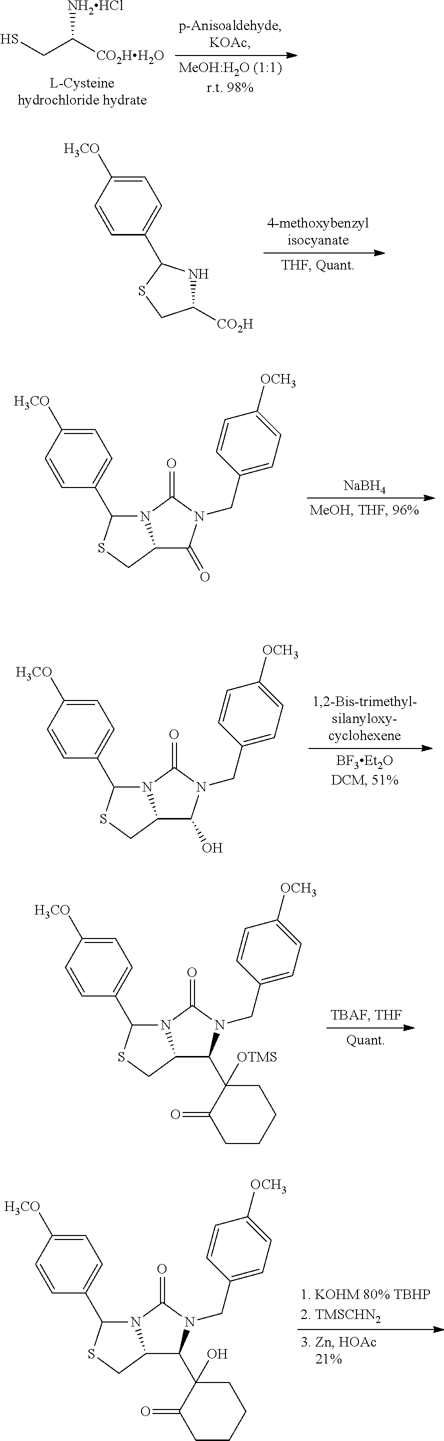

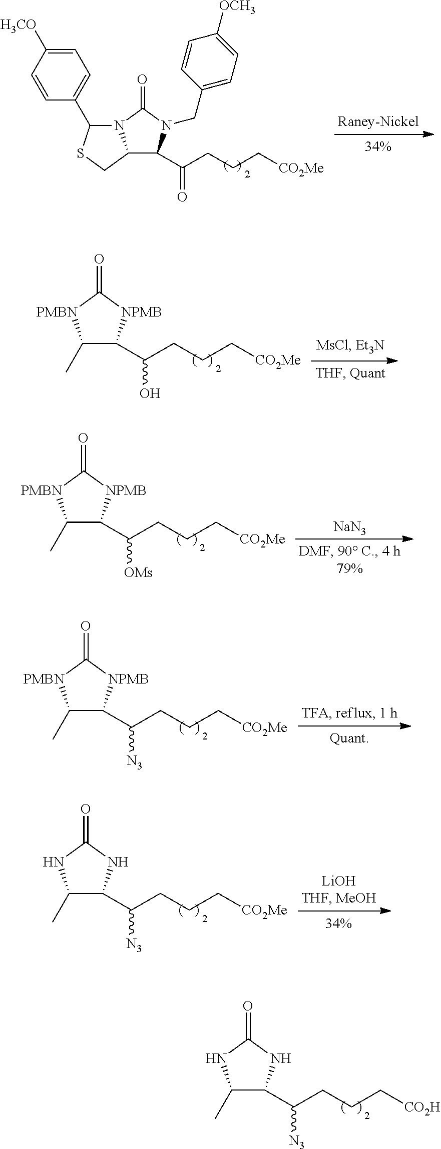

The properties of these analogues were then compared with a biotin analogue according to a first aspect of the present invention (namely, 2-Azidobiotin), native biotin and three further prior art biotin analogues, Iminobiotin, Ketone biotin and cis-propargyl biotin, the structures of which are given below:

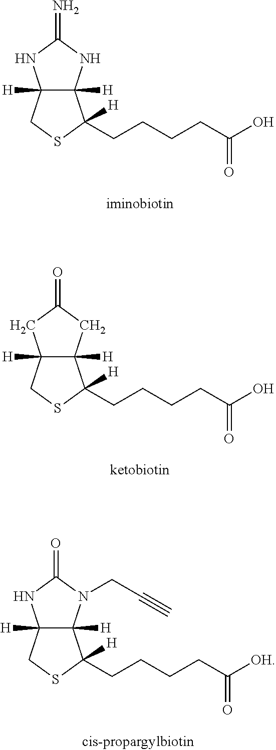

The properties of the biotin and its various analogues are summarised in the Table 2 below:

TABLE 2

| | | Substrate for | | | Binds to avidin |
|---|---|---|---|---|---|
| | Substrate | other biotin | | Bioorthogonally | once covalently |
| Ligand | for BirA | ligases | Binds to avidin | Functionalised? | modified |
| A biotin | Yes | Yes | Yes (K_{d} ~10⁻¹⁵M)¹ | No | N/A |
| B iminobiotin | No | No | Yes (K_{d} ~10⁻¹¹M)² | No | N/A |
| | | | pH dependent | | |
| C ketobiotin | Yes | ? | ? | Yes | No |
| | (modified) | | | | |
| D 8-azido- | No | Yes | Very weak | Yes | No |
| desthiobiotin | | | | | |
| E 6-azido- | No | ? | Very weak | Yes | No |
| desthiobiotin | | | | | |
| F cis- | No | Yes | No/very weak | Yes | No |
| propargylbiotin | | | | | |
| 2 Azidobiotin | Yes | ? | Yes (K_{d} ~10⁻⁷M) | Yes | Yes |

Thus, it can be seen that 6- and 8-Azidodesthiobiotin have low affinities for avidin and are not substrates for biotin ligase (BirA) from E. Coli. In contrast, the biotin analogue according to the present invention has medium affinity for avidin and has similar ligation kinetics to biotin with BirA from E. Coli. The ability of 2-Azidobiotin to bind with moderate-good affinity allows it to be used as an affinity purification tag either before the azide group has been modified or afterwards. The analogue may also be prepared easily from biotin in five steps. It therefore offers a useful new multipurpose tool for use in proteomics and biotechnology applications such as in studying histone biotinylation.

1. A biotin analogue comprising the ureido ring of natural biotin, optionally a modified thiophene ring and a modified sidechain having a functional end group selected from the group consisting of a carboxylic acid, aldehyde, alcohol, amine, thiol and halide, and at least one bio-orthogonally reactive chemical group located elsewhere in the sidechain, wherein the bio-orthogonally reactive chemical group is selected from the group consisting of an azide, an alkyne, an alkene, a heterocyclic group, a diene group and/or one or more heteroatoms selected from S, N, Se, P and

2-3. (canceled)

4. A biotin analogue as claimed in claim 1, wherein the reactive group is located on, or as part of, or in place of, a valeryl side chain of the biotin analogue.

5. A biotin analogue as claimed in claim 1, wherein the biotin analogue has the sulfur ring of the thiophene ring replaced with another group selected from the group consisting of CH₂, O, NH, and C═O.

6. (canceled)

7. A biotin analogue as claimed in 3 having the following general formula: 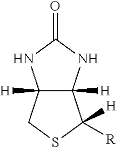
wherein R has a functional end group selected from the group consisting of a carboxylic acid, aldehyde, alcohol, amine, thiol and halide and includes at least one second functional group selected from the group consisting of an azide, an alkyne, an alkene, a diene, a heterocyclic ring and/or one or more heteroatoms selected from S, N, Se, P and O located elsewhere on the sidechain.

8. (canceled)

9. A biotin analogue as claimed in claim 7, wherein the bio-orthogonally reactive group is positioned at any one of positions 1 to 5 of the valeryl sidechain.

10. (canceled)

11. A biotin analogue as claimed in claim 7, wherein R is selected from the following side chains:

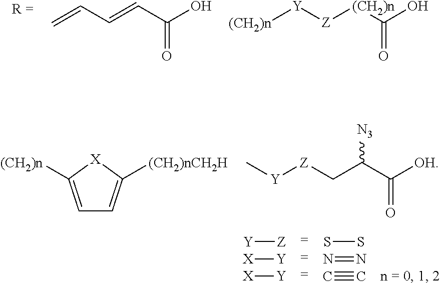

12. A biotin analogue as claimed in any one of claims 7, wherein the 5-carbon backbone of the valeryl sidechain of the analogue is maintained.

13. (canceled)

14. A biotin analogue as claimed in claim 7, wherein the backbone of the valeryl side chain includes one or more heteroatoms selected from the group consisting of sulphur, nitrogen, selenium, phosphorus or oxygen.

15. A biotin analogue having a structure selected from the group consisting of:

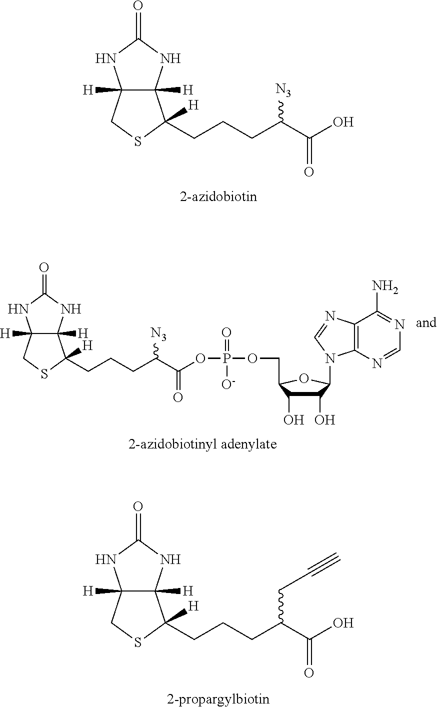

16-17. (canceled)

18. A biotin analogue having the structure: 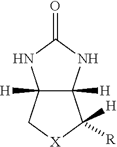
wherein X is CH2, O, NH or C═O and R has a functional end group and includes at least one second functional group selected from the group consisting of an azide, an alkyne, an alkene, a diene, a heterocyclic ring and/or one or more heteroatoms selected from S, N, Se, P and O located elsewhere on the sidechain.

19. A biotin analogue having the structure: 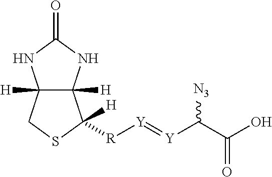
wherein X is CH2, O, NH or C═O and Y is N, CH or S.

20. A biotin analogue having the structure: 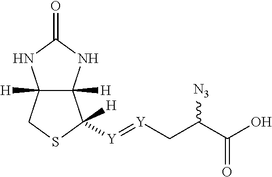
wherein X is CH2, O, NH or C═O and Y is N, CH or S.

21. (canceled)

22. A biotin analogue as claimed in claim 7 having the following general formula:

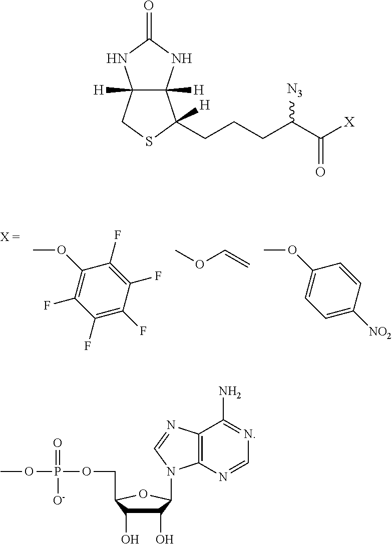

23-25. (canceled)

26. A specific target structure labelled with a biotin analogue as claimed in claim 1.

27-33. (canceled)

34. A method of labelling a target structure comprising a biotin analogue according to claim 1.

35-39. (canceled)
